(19) United States
(12) Patent Application Publication
(10) Pub. No.: US 2025/0145956 A1
(43) Pub. Date: May 8, 2025

(54) METHODS FOR IN VITRO EVALUATION USING FUNCTIONAL ENGINEERED THREE-DIMENSIONAL TISSUES WITH CIRCUMFERENTIAL OR HELICALLY ORIENTED TISSUE STRUCTURE

(71) Applicant: President and Fellows of Harvard College, Cambridge, MA (US)

(72) Inventors: John F. Zimmerman, Boston, MA (US); Huibin Chang, Cambridge, MA (US); Kevin Kit Parker, Cambridge, MA (US)

(21) Appl. No.: 18/684,234

(22) PCT Filed: Jul. 1, 2022
(86) PCT No.: PCT/US2022/036011

§ 371 Date: Feb. 16, 2024

Related U.S. Application Data
(60) Provisional application No. 63/234,287, filed on Aug. 18, 2021.

(51) Int. Cl. C12N 5/077; G01N 33/50

(52) U.S. Cl. CPC C12N 5/0657; G01N 33/5082; C12N 2513/00

(57) ABSTRACT

Methods of evaluating functional activity of an engineered three-dimensional tissue, methods of identifying a compound that modulates tissue function, and methods for identifying a compounds that is useful for treating or preventing a disease affecting tissue function are provided herein. Some methods include providing or obtaining a three-dimensional tissue scaffold defining a lumen or a cavity, the tissue scaffold comprising one or more polymeric fibers each having a micron-scale or nanometer-scale diameter, at least some of the one or more polymeric fibers encircling the lumen or cavity at a helical angle with respect to a longitudinal axis of the lumen or cavity, at an azimuthal orientation with respect to the longitudinal axis of the lumen or cavity, or at both.

### GOVERNMENT SUPPORT

This invention was made with Government support under grant number UH3TR000522 awarded by National Institutes of Health and under grant number 1-UG3-HL-141798-01 awarded by National Center for Advancing Translational Sciences. The government has certain rights in the invention.

### TECHNICAL FIELD

Embodiments of the disclosure relate to methods of evaluating functional activity of an engineered three-dimensional tissue, methods of identifying a compound that modulates tissue function, and methods for identifying a compounds that is useful for treating or preventing a disease affecting tissue function.

### BACKGROUND OF THE INVENTION

Identification and evaluation of new therapeutic agents or identification of suspect disease associated targets typically employ animal models which are expensive, time consuming, require skilled animal-trained staff and utilize large numbers of animals. In vitro alternatives have relied on the use of conventional cell culture systems which are limited in that they do not allow the three-dimensional interactions that occur between cells and their surrounding tissue. This is a serious disadvantage as such interactions are well documented as having a significant influence on the growth and activity of cells in vivo since in vivo cells divide and interconnect in the formation of complex biological systems creating structure-function hierarchies that range from the nanometer to meter scales.

Accordingly, there is a need for improved methods and systems that may be used for determining the effect of a test compound on biological relevant parameters in order to enhance and speed-up the drug discovery and development process.

### SUMMARY OF THE INVENTION

Some embodiments of the present invention include methods of evaluating functional activity of an engineered three-dimensional tissue, methods of identifying a compound that modulates tissue function, and methods for identifying a compounds that is useful for treating or preventing a disease affecting tissue function.

In one aspect, the present invention provides a method which includes providing or obtaining a three-dimensional tissue scaffold defining a lumen or a cavity, the tissue scaffold comprising one or more polymeric fibers each having a micron-scale or nanometer-scale diameter, at least some of the one or more polymeric fibers encircling the lumen or cavity at a helical angle with respect to a longitudinal axis of the lumen or cavity, at an azimuthal orientation with respect to the longitudinal axis of the lumen or cavity, or at both; providing or obtaining cells growing on or in the three-dimensional tissue scaffold to form a three-dimensional tissue defining the lumen or the cavity; affixing a first portion of the three-dimensional tissue to a support where the cells are grown on or in the three-dimensional tissue scaffold to form the three-dimensional tissue before the three-dimensional tissue is affixed to the support, or affixing a first end of the three-dimensional tissue scaffold to a support where the cells are grown on or in the three-dimensional tissue scaffold to form the three-dimensional tissue after the three-dimensional tissue scaffold is affixed to the support; and measuring rotational displacement of at least a second portion of the three-dimensional tissue relative to the support over a period of time caused by functional activity of three-dimensional tissue or measuring strain of at least a second portion of the three-dimensional tissue relative to the support over a period of time caused by functional activity of the three-dimensional tissue.

In one embodiment, the cells comprise at least one of vascular smooth muscle cells, cardiac myocytes, skeletal muscle cells, uterine smooth muscle cells, intestinal smooth muscle cells, myofibroblasts, airway smooth muscle cells, osteoblasts, myoblasts, neuroblasts, fibroblasts, glioblasts, germ cells, trophoblasts, lymphocytes, intestinal crypt cells, intestinal villi cells, chondrocytes, keratinocytes, connective tissue cells, glial cells, epithelial cells, endothelial cells, vascular endothelial cells, hormone-secreting cells, neural cells, and cells that will differentiate into muscle cells.

In one embodiment, the cells comprise muscle cells.

In one embodiment, the cells comprise cardiac muscle cells.

In one embodiment, rotational displacement of at least a second portion of the three-dimensional tissue relative to the support over a period of time caused by functional activity of three-dimensional tissue is measured.

In one embodiment, the strain of at least the second portion of the three-dimensional tissue relative to the support over the period of time caused by functional activity of the three-dimensional tissue is measured; and wherein measuring the strain of at least the second portion of the three-dimensional tissue relative to the support over the period of time caused by functional activity of the three-dimensional tissue comprises mapping strain of the at least the second portion of the three-dimensional tissue over the period of time.

In one embodiment, comprise vascular epithelial cells.

In one embodiment, measuring rotational displacement of at least the second portion of the three-dimensional tissue relative to the support over the period of time or measuring strain of at least the second portion of the three-dimensional tissue relative to the support over the period of time comprises obtaining images of at least the second portion of the three-dimensional tissue over the period of time.

In one embodiment, the images of at least the second portion of the three-dimensional tissue include images of a two-dimensional perimeter or edge of the three-dimensional tissue.

In one embodiment, measuring rotational displacement of at least the second portion of the three-dimensional tissue relative to the support over the period of time further comprises measuring a change in an orientation of the two-dimensional perimeter or edge of the three-dimensional tissue from the images over the period of time.

In one embodiment, measuring rotational displacement of at least the second portion of the three-dimensional tissue relative to the support over the period of time or measuring strain of at least the second portion of the three-dimensional tissue relative to the support over the period of time further comprises determining changes in position of the two-dimensional perimeter or edge of the three-dimensional tissue from the images over the period of time.

In one embodiment, the first portion of the three-dimensional tissue is disposed at a first end of the three-dimensional tissue and the at least the second portion of the three-dimensional tissue is disposed at a second end of the three-dimensional tissue opposite the first end of the three-dimensional tissue that is affixed to the support.

In one embodiment, the method further comprises exposing the three-dimensional tissue scaffold to a solution containing fiducial markers such that the fiducial markers adhere to the three-dimensional tissue scaffold, or exposing the three-dimensional tissue to a solution containing fiducial markers that adhere to the three-dimensional tissue; and wherein measuring rotational displacement of at least a second portion of the three-dimensional tissue relative to the support over a period of time or measuring strain of at least the second portion of the three-dimensional tissue relative to the support over the period of time comprises performing imaging of the fiducial markers in or on the at least a second portion of the three-dimensional tissue over the period of time.

In one embodiment, the fiducial markers are optical fiducial markers comprising fluorescent beads or fluorescent particles that adhere to the three-dimensional scaffold.

In one embodiment, the optical fiducial markers comprise an immunofluorescent stain.

In one embodiment, the fiducial markers comprise one or more of reflective beads, reflective particles, optically scattering beads, and optically scattering particles.

In one embodiment, measuring rotational displacement of at least a second portion of the three-dimensional tissue relative to the support over a period of time or measuring strain of at least the second portion of the three-dimensional tissue relative to the support over the period of time further comprises performing strain mapping based on the imaging.

In one embodiment, measuring rotational displacement of at least the second portion of the three-dimensional tissue relative to a support over the period of time or measuring strain of at least the second portion of the three-dimensional tissue relative to the support over the period of time is via one or more strain sensors attached to the three-dimensional tissue.

In one embodiment, the method further comprises stimulating the three-dimensional tissue before, during, or before and during at least some of the measurement of the rotational displacement of at least the second portion of the three-dimensional tissue relative to the support over the period of time the measurement of the strain of at least the second portion of the three-dimensional tissue relative to the support over the period of time to cause a functional response in the three-dimensional tissue, to initiate the functional activity of the three-dimensional tissue, or to affect the functional activity of the three dimensional tissue.

In one embodiment, the method is a method of evaluating the functional response or functional activity of the three-dimensional tissue to the stimulation.

In one embodiment, the functional response is a biomechanical activity, an electrophysiological activity, or both.

In one embodiment, the method is a method of evaluating the functional activity of the three-dimensional tissue.

In one embodiment, the method is a method for identifying a compound that modulates tissue function; and wherein the method further comprises: contacting the three-dimensional tissue structure with a test compound, wherein measuring rotational displacement of at least a second portion of the three-dimensional tissue with respect to the support over a period of time or measuring strain of at least the second portion of the three-dimensional tissue relative to the support over the period of time includes: measuring a first rotational displacement of the at least the second portion of the three-dimensional tissue relative to the support in the presence of the test compound over a first period of time or measuring a first strain of at least the second portion of the three-dimensional tissue relative to the support in the presence of the test compound over a first period of time; and measuring a second rotational displacement of the at least the second portion of the three-dimensional tissue relative to the support in the absence of the test compound over a second period of time or measuring a second strain of at least the second portion of the three-dimensional tissue relative to the support in the absence of the test compound over a second period of time; and comparing the first rotational displacement of at least the second portion of the three-dimensional tissue or the first strain of at least the second portion of the three-dimensional tissue with the second rotational displacement of the at least the second portion of the three-dimensional tissue or the second strain of at least the second portion of the three-dimensional tissue, wherein a modulation of the first rotational displacement or the first strain in the presence of the test compound as compared to the second rotational displacement or the second strain in the absence of the test compound indicates that the test compound modulates tissue function, thereby identifying a compound that modulates tissue function.

In one embodiment, the method is a method for identifying a compound that is useful for treating or preventing a disease affecting tissue function, and wherein the method further comprises: contacting the three-dimensional tissue structure with a test compound, wherein measuring rotational displacement of the at least a second portion of the three-dimensional tissue with respect to the support over a period of time or measuring strain of at least the second portion of the three-dimensional tissue relative to the support over the period of time includes: measuring a first rotational displacement of the at least the second portion of the three-dimensional tissue relative to the support in the presence of the test compound over a first period of time or measuring a first strain of at least the second portion of the three-dimensional tissue relative to the support in the presence of the test compound over a first period of time; and measuring a second rotational displacement of the at least the second portion of the three-dimensional tissue relative to the support in the absence of the test compound over a second period time or measuring a second strain of at least the second portion of the three-dimensional tissue relative to the support in the absence of the test compound over a second period of time; and comparing the first rotational displacement of at least the second portion of the three-dimensional tissue or the first strain of at least the second portion of the three-dimensional tissue with the second rotational displacement of the at least the second portion of the three-dimensional tissue or the second strain of at least the second portion of the three-dimensional tissue, wherein a modulation of the first rotational displacement or the first strain in the presence of the test compound as compared to the second rotational displacement or the second strain in the absence of the test compound indicates that the test compound modulates tissue function, thereby identifying a compound useful for treating or preventing a disease affecting tissue function.

In some embodiments, the three-dimensional tissue is or includes a tissue engineered ventricle or a model of a ventricle, wherein the method also includes determining one or more ejection fractions for the three-dimensional tissue over time based on the measured rotational displacement of at least the second portion of the three-dimensional tissue relative to the support over the period of time, or based on the measured strain of at least the second portion of the three-dimensional tissue relative to the support over the period of time

In one embodiment, the disease is a muscular disease, pericarditis, endocarditis, cardiac fibrosis, or an arterial disease.

In one embodiment, the muscular disease is hypertension structural arrhythmia, ischemia, an inherited heart conditions, pulmonary valve regurgitation, or mitral valve regurgitation.

In one embodiment, the tissue function is a biomechanical activity.

In one embodiment, the biomechanical activity is one or more of contractility, cell stress, cell swelling, and rigidity.

In one embodiment, the biomechanical activity is one or more of stem cell activation, stem cell maturation, tissue morphogenesis, and tissue remodeling.

In one embodiment, the tissue function is an electrophysiological activity.

In one embodiment, the electrophysiological activity is a voltage parameter selected from the group consisting of action potential, action potential duration (APD), conduction velocity (CV), refractory period, wavelength, restitution, bradycardia, tachycardia, reentrant arrhythmia, and a calcium flux parameter.

In one embodiment, the calcium flux parameter comprises one or more of intracellular calcium transient, transient amplitude, rise time (contraction), decay time (relaxation), total area under the transient (force), restitution, focal, and spontaneous calcium release.

In one aspect, the present invention provides a method which includes providing or obtaining a three-dimensional tissue scaffold defining a lumen or a cavity, the tissue scaffold comprising one or more polymeric fibers each having a micron-scale or nanometer-scale diameter, at least some of the one or more polymeric fibers encircling the lumen or cavity at a helical angle with respect to a longitudinal axis of the lumen or cavity, at an azimuthal orientation with respect to the longitudinal axis of the lumen or cavity, or both; providing or obtaining cells growing on or in the three-dimensional tissue scaffold to form a three-dimensional tissue defining the lumen or the cavity; exposing the three-dimensional scaffold to a medium including fiducial markers; obtaining images of an area including or adjacent to an opening of the lumen or cavity over a period of time; performing particle imaging velocimetry on fiducial markers in the images of the area including or adjacent to the opening of the lumen or cavity; and determining a volume, mass flux, or velocity of fluid flow out of or into the opening due to or affected by a functional activity of the three-dimensional tissue based on the particle imaging velocimetry.

In one embodiment, the cells comprise at least one of vascular smooth muscle cells, cardiac myocytes, skeletal muscle cells, uterine smooth muscle cells, intestinal smooth muscle cells, myofibroblasts, airway smooth muscle cells, osteoblasts, myoblasts, neuroblasts, fibroblasts, glioblasts, germ cells, trophoblasts, lymphocytes, intestinal crypt cells, intestinal villi cells, chondrocytes, keratinocytes, connective tissue cells, glial cells, epithelial cells, endothelial cells, vascular endothelial cells, hormone-secreting cells, neural cells, and cells that will differentiate into muscle cells.

In one embodiment, the cells comprise muscle cells.

In one embodiment, the cells comprise cardiac muscle cells.

In one embodiment, the cells comprise vascular epithelial cells.

In one embodiment, the fiducial markers are suspended in the medium.

In one embodiment, the fiducial markers comprise or are attached to beads or particles.

In one embodiment, the beads or particles are neutrally buoyant in the medium.

In one embodiment, the fiducial markers are fluorescent.

In one embodiment, the beads or particles are reflective or optically scattering; and wherein the imaging, at least in part, is of darkfield scattering.

In one embodiment, the method further comprises stimulating the three-dimensional tissue before, during, or before and during obtaining the images over the period of time, to initiate the functional activity of the three-dimensional tissue, or to affect the functional activity of the three-dimensional tissue.

In one embodiment, the method is a method of evaluating the functional activity of the three-dimensional tissue in response to the stimulation.

In one embodiment, the functional activity is a biomechanical activity, an electrophysiological activity, or both.

In one embodiment, the method is a method of evaluating the functional activity of the three-dimensional tissue.

In one embodiment, the method is a method for identifying a compound that modulates tissue function; wherein the method further comprises contacting the three-dimensional tissue structure with a test compound; wherein obtaining images of the area including or adjacent to the opening of the lumen or cavity over a period of time includes: obtaining first images of the area in the presence of the test compound over a first period of time; and obtaining second images of the area in the absence of the test compound over a second period of time; wherein determining a volume, mass flux, or velocity of fluid flow out of or into the opening due to or affected by a functional activity of the three-dimensional tissue based on the particle imaging velocimetry includes: determining a first volume, mass flux, or velocity of fluid flow out of or into the opening during the first period of time; and determining a second volume, mass flux, or velocity of fluid flow out of or into the opening during the second period of time; and wherein the method further comprises comparing the first volume, mass flux, or velocity of fluid flow with the second volume, mass flux, or velocity of fluid flow, wherein a modulation of the first volume, mass flux, or velocity of fluid flow in the presence of the test compound as compared to the second volume, mass flux, or velocity of fluid flow in the absence of the test compound indicates that the test compound modulates the tissue function.

In some embodiments, the three-dimensional tissue is or includes a tissue engineered ventricle or a model of a ventricle, and the method also includes determining one or more ejection fractions for the three-dimensional tissue based, at least in part, on the determined volume, mass flux, or velocity of fluid flow out of or into the opening.

In one embodiment, the method is a method for identifying a compound that is useful for treating or preventing a disease affecting tissue function; wherein the method further comprises contacting the three-dimensional tissue structure with a test compound; wherein obtaining images of the area including or adjacent to the opening of the lumen or cavity over a period of time includes: obtaining first images of the area in the presence of the test compound over a first period of time; and obtaining second images of the area in the absence of the test compound over a second period of time; wherein determining a volume, mass flux, or velocity of fluid flow out of or into the opening due to or affected by a functional activity of the three-dimensional tissue based on the particle imaging velocimetry comprises: determining a first volume, mass flux, or velocity of fluid flow out of or into the opening during the first period of time; and determining a second volume, mass flux, or velocity of fluid flow out of or into the opening during the second period of time; and wherein the method further comprises comparing the first volume, mass flux, or velocity of fluid flow and the second volume, mass flux, or velocity of fluid flow, wherein a modulation of the first volume, mass flux, or velocity of fluid flow as compared to the second volume, mass flux, or velocity of fluid flow indicates that the test compound modulates tissue function, thereby identifying a compound useful for treating or preventing a disease affecting tissue function.

In one embodiment, the disease is a muscular disease, a vascular disease, pericarditis, endocarditis, or cardiac fibrosis.

In one embodiment, the muscular disease is hypertension structural arrhythmia, ischemia, an inherited heart condition, pulmonary valve regurgitation, or mitral valve regurgitation.

In one embodiment, the tissue function is a biomechanical activity.

In one embodiment, the biomechanical activity is one or more of contractility, cell stress, cell swelling, and rigidity.

In one embodiment, the biomechanical activity is one or more of stem cell activation, stem cell maturation, tissue morphogenesis, and tissue remodeling.

In one embodiment, the tissue function is an electrophysiological activity.

In one embodiment, the electrophysiological activity is a voltage parameter selected from the group consisting of action potential, action potential duration (APD), conduction velocity (CV), refractory period, wavelength, restitution, bradycardia, tachycardia, reentrant arrhythmia, and/or a calcium flux parameter, e.g., intracellular calcium transient, transient amplitude, rise time (contraction), decay time (relaxation), total area under the transient (force), restitution, focal and spontaneous calcium release.

In one embodiment, the calcium flux parameter comprises one or more of intracellular calcium transient, transient amplitude, rise time (contraction), decay time (relaxation), total area under the transient (force), restitution, focal, and spontaneous calcium release.

In one embodiment, the three-dimensional tissue structure comprises an engineered tissue ventricle.

In one embodiment, the three-dimensional tissue structure comprises an engineered tissue heart.

In one embodiment, the three-dimensional tissue structure comprises one or more of a heart, an artery, a blood vessel, a lymph node, a lymphatic vessel, an intestine, a myometrium, and an inner layer of a tongue.

In one embodiment, the three-dimensional tissue scaffold includes: a first layer in which a first portion of the one or more polymeric fibers encircle the longitudinal axis of the lumen at a first helical angle with respect the longitudinal axis of the lumen or cavity; and a second layer in which a second portion of the one or more polymeric fibers encircle the longitudinal axis of the lumen or cavity a second helical angle with respect to the longitudinal axis of the lumen or cavity, wherein a difference between the first helical angle and the second helical angle falls is less than 90°.

In one embodiment, the difference between the first angle and the second angle falls in a range of 300 to 60°.

In one embodiment, the difference between the first helical angle and the second helical angle falls in a range of 520 to 580 or a range of 32.5° to 35.8°.

In one embodiment, providing or obtaining the three-dimensional tissue scaffold comprises: rotating a reservoir holding a material comprising a polymer about a rotation axis to eject at least one jet of material from at least one orifice defined by an outer sidewall of the reservoir; directing at least one flow of gas through a portion of the reservoir radially inward of the outer sidewall, the at least one flow of gas directed from an upstream first end of the reservoir to a downstream second end of the reservoir during rotation of the reservoir and ejection of the at least one jet of the material to form at least one micron or nanometer dimension polymeric fiber, the at least one flow of gas entraining the at least one micron or nanometer dimension polymeric fiber and forming a focused fiber deposition stream of the at least one micron or nanometer dimension polymeric fiber in a first direction, the first direction having an orientation of within 45 degrees of the rotation axis of the reservoir; and collecting the focused fiber deposition stream on a target surface that is being rotated about a second rotation axis, the target surface having a shape corresponding to at least a portion of the three-dimensional tissue scaffold.

In one embodiment, providing or obtaining the three-dimensional tissue scaffold comprises further comprises changing the rotation axis of the target surface to a third rotation axis different than the second rotation axis and collecting the focused fiber deposition stream on the target surface when the target surface is being rotated about the third rotation axis, wherein an angle between the second rotation axis and the third rotation axis falls in a range of 2° to 90°.

Other features and advantages of the invention will be apparent from the following detailed description and claims.

### BRIEF DESCRIPTION OF THE DRAWINGS

FIG. 1A schematically depicts a perspective view of a three-dimensional tissue scaffold defining a cavity including polymeric fibers that encircle the cavity at a helical angle with respect to a longitudinal axis of the cavity in accordance with some embodiments.

FIG. 1B schematically depicts a perspective view of a three-dimensional tissue scaffold defining a cavity including polymeric fibers that encircle the cavity with an azimuthal or cylindrical orientation with respect to a longitudinal axis of the cavity in accordance with some embodiments.

FIG. 1C schematically depicts a perspective view of a three-dimensional tissue scaffold defining a lumen including polymeric fibers that encircle the lumen at a helical angle with respect to a longitudinal axis of the lumen in accordance with some embodiments.

FIG. 1D schematically depicts a perspective view of a three-dimensional tissue scaffold defining a lumen including polymeric fibers that encircle the lumen with an azimuthal or cylindrical orientation with respect to a longitudinal axis of the cavity in accordance with some embodiments.

FIGS. 2A-2F. (A) Graph of throughput versus feature size for 3D printing via extrusion showing that 3D printing throughput scales linearly with feature size, while organs scale volumetrically, creating a fundamental limit in 3D printing speed (B) Schematically depicting focused rotary jet spinning that uses a focused air stream to structure fibers into a confined and aligned pattern. This approach separates fiber formation and patterning into two distinct phases. (C) Differential contrast projection image of a fiber stream from a focused rotary jet spinning system (maximal projection, scale 5 cm). (D) Upper image showing fiber profiles collected on metallic rods at different points in a focused rotary jet spinning stream showing focusing (upper, scale 50 mm), and lower graph of fiber profile versus collector distance with corresponding thickness distributions (lower) measured as a function of distance between the collector and spinneret (profiles amplified 25× for clarity, unbroken line-profile, dashed line-Gaussian fit). (E) Image showing conformal deposition of fibers onto a rodent scale ventricle model (lower, scale 5 mm). (F) Electron micrograph of polycaprolactone nanofibers showing aligned micro/nanofiber formation (mean fiber diameter ˜900 nm, scale 5 μm).

FIGS. 3A-3G. Fiber Alignment Controls Tissue Organization. (A) Schematic of fiber collection (top left), and graph or orientation order parameter (OOP) versus collection angle (θ) showing that the collection angle (θ) dictates the relative alignment of deposited fibers fiber, and. SEM micrographs (lower) (0°, 60°, and 90°, scale bars 5 μm), with corresponding 2D Fourier transforms (inset), indicating the degree of alignment. Scale bars are 5 μm. Orientation Order Parameter (OOP) indicates the relative alignment across multiple production runs (upper right). (B) Schematic depicting how rich patterning with controlled orientation can be achieved by manipulating the deposition angle and position of the target (left). Image of cross-section of fiber matt with the corresponding alignments, reconstructed using micro-CT (right, scale bar 100 μm). (C) Schematic depicting fabrication approach for helically aligned cylinders with helical angle α, and (D) corresponding free-body diagram of a released fiber structure (T-Tension, H-Hoop Strain, A-Axial Strain). (E) Reconstructed tomograph of a helically aligned scaffold using micro-CT, with (F) inset from highlighted region (scalebar 200 μm). (G) SEM micrograph of gelatin nanofibers from circumferentially (i., α=0°) and (iii., α=45°) helically aligned cylinders, with corresponding immunofluorescent staining of cardiomyocytes (ii & vi) (NRVM, Medium Gray-Dapi, white-Sarcomeres, Dark Gray-F Actin), showing that fibers control subsequent cell infiltration and alignment (scale bars 50 μm).

FIGS. 4A-4I. Functional Tissue Engineered Ventricle Models (A) Schematic illustration of the heart. (B) Image of scale model of the left ventricle made with gelatin nanofibers, seeded with cardiomyocytes (scale bar, 5 mm) (C). Immunofluorescent micrograph of cardiomyocytes on the ventricle scaffold (Dapi-Medium Gray, sarcomeres-White, Light Gray, f-actin-Dark Gray, scale bar 10 μm) (D) Schematic diagram of a cylindrically aligned (CA) fiber ventricle (upper) and helically aligned (HA) fiber ventricle (lower), showing differences in wall displacement before (gray outer) and after (dark gray inner) contraction. (E) Map of strain generated during peak contraction in CA (upper) and HA (lower) ventricle models (scale bar 2 mm). (F) Images showing isochrones (upper) with corresponding still-frames (lower), indicating calcium transience along an extended ventricle surface, showing increased transverse wave propagation for HA scaffolds. Point stimulated at apex. (i.-CA, ii.-HA, scale bars 5 mm). (G) Particle imaging velocimetry (PIV) velocity fields taken from the basal opening of a HA ventricle scaffold during peak systole (upper, t=0.3 s) and diastole (lower, t=0.7 s). (H) Graph of representative measurements of the instantaneous mass flux in the region of interest as function of contraction time. (I) Graph of ensemble measurements of ejection fraction (EF) for CA and HA ventricle scaffolds (n>8 ventricles for each angle).

FIGS. 5A-5G. Multiscale Heart Scaffolds. (A) Schematic depicting simplified tri-layered Duel Chamber Ventricle (DCV) design mimicking the native ECM alignment of the heart. (B) Schematic depicting a four-step manufacturing of DCV model, with an image of the resulting structure (scale bar, 5 mm). (C) Perspective, top, and side images from a micro-CT characterization of the resulting DCV structure showing axes of the intraventricular septum (highlighted in medium gray), showing tri-layer alignments from the respective regions (insets) (scale bars, 100 μm). (D) Images of micro-CT high magnification windows for an area of the intraventricular septum indicated with a circle in the top and side images in (C), showing tri-layer alignments from the respective regions (insets) (scale bars, 100 μm). (E) Immunofluorescent micrographs of human derived cardiomyocytes taken from an excised region of the DCV's left ventricle wall, showing aligned cardiac tissue (Medium Gray-DAPI, Light Gray-Sarcomeres, Dark Gray-Actin, Scale bar, 100 μm) (F) Calcium signaling map at different time points along an excised region of DCV's left ventricle, showing sustained calcium wave propagation (scale bar 2 mm), (G) Perspective image of a full-scale four chambered human heart model composed of micron/nanoscale fibers.

FIGS. 6A-6F. Design of the Focused Rotary Jet Spinning Platform (A) Exploded perspective view schematically depicting components of the focused rotary jet spinning system or platform. The spinning platform consists of a spinneret, an air blower, a motor, and tubes that feed the polymer solution into the spinneret. Compressed air is fed into the air blower, while the spinneret is hollowed out in the center to allow air to blow out. The air blower has three nozzles, with the three streams of air merging into a single air jet in front of the spinneret. The number of air nozzles matches the number of spokes on the spinneret so that as the spinneret spins, the air jet pulsates, maintaining the same directionality. (B) Schematic depicting perspective section view of air-blower. (C) Schematic depicting perspective section view of spinneret. (D) Photograph with front perspective view of assembled blower, tubes, motor, and spinneret. (E) Photograph with rear perspective view of assembled blower, tubes, motor, and spinneret. (F) Photograph of focused rotary jet spinning platform with syringe pump control panel, motor control panel, and solution in syringe.

FIGS. 7a-7d. Increased Major Axis Shortening in Helically Aligned Ventricles. (a) Photograph of a representative ventricle scaffold, visually isolated for thresholding (b) Representative perimeter outlines for circumferentially and helically aligned ventricles during contraction (red) and relaxation (black). Magnified insets, taken from the highlighted basal (B) and apical (A) regions, showing minimal apical and increased basal changes in the circumferentially aligned ventricles. (c) Schematic diagram showing quantification method of axial shorting using elliptical fitting of thresholded ventricle areas, to obtain the semi-major (Rₘₐⱼ) and semi-minor axis (Rₘᵢₙ) lengths. (d) Plot of average change (ΔR) in in the semi-major and semi minor axis for circumferentially and helically aligned ventricles, normalized by the total area change, showing increased apical shortening in the helically aligned case. n≥5 ventricles for each test condition. * indicates p<0.05 as determined by a pairwise students T-test.

FIGS. 8A-8C. Calcium Wave Propagation in 2D Fiber Tissues. (A) Micrograph of 2D fiber scaffold seeded with NRVMs (scale 2 mm) (B) Bright field micrograph super imposed with calcium signal, showing wave front propagation at different time points after stimulation (scale 2 mm). (C) Isochrone of calcium wave propagation, showing the conductance velocity in the longitudinal (V_{L}) and transverse (V_{T}) direction of fiber alignments (upper). Calcium signal intensity given as a function of time for each of the four corners (lower).

FIGS. 9A-9E. Modeling Ventricle Twist (A) Ventricle scaffold sutured at the base and suspended, allowing free rotation about the apex (XZ Side view, scale bar, 10 mm) (B) Bottom-up view of a suspended ventricle scaffold for quantifying rotation (XY Bottom-Up View) (C) Schema for mapping rotational displacement based on perimeter or edge boundary. Scaffolds are thresholded, and the boundary is fit to an ellipse. Changes in the angle of the ellipses major axis are recorded as rotational displacements. (D). Time course of rotational displacement for representative HA (α=30°) and CA (α=0°) ventricles, demonstrating apical twist in the HA case (1 Hz, 10 v Field Stimulation). (E) Plot of average rotational displacement as a function of ventricle alignment. (n=7 ventricles each) (statistically significant difference, p<0.005).

FIGS. 10A-10F. Particle Imaging Velocimetry of Helically Aligned Ventricles. (A) Bright field micrograph of a helically aligned ventricle, resting against the base of a petri dish to prevent travel (scale bar 2 mm). Increased magnification micrograph of the basal region of circumferentially (α=0°)(B) and helically (α=30°) (C) aligned ventricle scaffolds (left), with the corresponding trace showing the flow path of fluorescent bead's (right) (max projection taken over 4 s, scale bars-2 mm). Flow paths were generally increased for helically aligned scaffolds. PIV was captured over the basal region of interest (ROI) highlighted in yellow. (D) Schematic showing the relative direction of the fluid velocity fields. (E) Normalized sum of the longitudinal components, U, of the fluid velocity taken over the highlighted ROI continuous line. Velocity components were fit using a sin function (dotted line) to determine the period and phase of contraction. (F) Graph of phase averaged single contraction cycle, obtained by averaging each frame over the entire collection period.

FIGS. 11A-11D. Ventricle Pressure Volume Measurements. (A.) Darkfield micrograph of a circumferentially aligned gelatin nanofiber ventricle scaffold seeded with NRVMs, sutured to a Pressure-Volume catheter. (Scale bar 2 mm). (B) Schematic illustration of catheter's localization within the ventricle scaffold. (C) Trace of Pressure-Volume changes taken during spontaneous cardiac contraction. Values normalized to ambient conditions. (D) Graph of pressure-Volume loop, time averaged over the complete trace. As the model systems did not contain valves, the resulting measurement lacked any indicators of isovolumetric relaxation, as is consistent with valvular defects.

FIGS. 12A-12D. Four-step manufacturing of the tri-layered dual chambered ventricle model. (A) A 3D printed target with the shape of the left ventricle is pointed into the fiber stream at 45°. The target rotates counterclockwise at 2,000 RPM to collect the helically aligned inner layer. (B) The target is turned vertical and rotates at 10,000 RPM to collect the circumferentially aligned middle layer. (C) A 3D printed target with the shape of the right ventricle is pointed into the fiber stream at 45°. The target rotates counterclockwise at 2,000 RPM to collect the helically aligned inner layer. (D) The two targets with the deposited fiber are taped together and pointed into the fiber stream at 45°. The combined target rotates clockwise at 2,000 RPM to collect the helically aligned outer layer.

FIGS. 13A-13C. Human stem cell differentiation into cardiomyocytes. (A) Schematic overview of the fifteen day differentiation protocol, including sodium lactate purification steps. (B) Brightfield micrographs of stem cell differentiation, showing changes in cellular morphology at Day 0, Day 5 and Day 11 in culture. (C) Representative immunofluorescent micrograph of stem-cell derived cardiomyocytes after fifteen days in culture, showing the formation of aligned cardiomyocyte bundles expressing well-organized sarcomeric α-actinin (scale bar, 50 μm. Dapi-Medium Gray, F-Actin-Dark Gray, sarcomeric α-actinin—Light Gray).

FIGS. 14A-14B. Transmural fiber orientation through an equatorial section of an adult rat left ventricle wall. (A) Adult rat heart shown before (left) and after (right) it was sectioned at the equator. (B) Cardiomyocyte fibers in the left ventricle wall immunostained for nuclei (DAPI), F-actin (phalloidin), and α-actinin, showing fiber orientation from the endocardium (endo) to the epicardium (epi). Scale bars are 100 μm (top) and 30 μm (bottom).

FIGS. 15a-15f. Three Dimensional In Vitro Nanofiber Scaffold model of Hypertensive Remodeling. (a) Schematic illustration of focused rotary jet spinning (FRJS) for manufacturing aligned ventricle scaffolds. (b) Example photographs of conical collection mandrel before and after FRJS. (c) Photograph of resulting ventricle scaffold seeded with cardiomyocytes (left) and immunofluorescent micrograph showing aligned cardiac tissue (Inset right). (d) Schematic depicting non-specific adhesion of fluorescent beads to the ventricle surface. (e) Fluorescence image of ventricle with adhered beads. (f) Strain mapping during peak systole based on fluorescence imaging of the ventricle with adhered fluorescent beads.

FIGS. 16A.-16E. Fibroblast/NRVM Co-Cultured Ventricles. (A) Immunofluorescence micrographs of NRVMs, staining for sarcomeric α-actinin, and α-smooth muscle actin, indicating cardiac and fibroblast cells respectively. Images taken of cardiomyocyte co-cultures (i.) pre-plating and (ii.) post-plating, to filter out fibroblasts. (scale bars, 50 μm top, 25 μm bottom) (B) Deformation mapping of single chamber ventricles, cultured with NRVMS (i. 5 million cells, Post-plating population, NRVMs), and fibroblast/NRVM samples (ii. 5 million cells, Pre-plating cell population, Fibro I) (iii. 7.5 million cells, Pre-plating cell population, Fibro II), controlling for total cell population, and cardiac cell population respectively (scale bars, 2 mm). (C) Example PIV velocity taken at the base of the ventricle for NRVM (i.) and fibroblast/NRVM (ii.) ventricles at peak systole, showing differences in ejection velocities. (scale bars, 2 mm). (D) Instantaneous mass flux from representative ventricles, showing reduced output for fibroblast/NRVM co-cultures. (E) Ejection fraction box plot for single chamber ventricle samples, showing reduced ejection fractions for fibroblast/NRVM co-culture samples (n=3-4 ventricles each)(box plots given in quartiles).

FIGS. 17A.-17E. Ventricle Construct Cell Coverage. (A) Photograph of a ventricle construct, pressed flat between two panes of glass. (B) Confocal fluorescent tile scan of neonatal rat ventricular myocytes (NRVMs) (i.), acquired over the ventricle region indicated. The tile scan, reconstructed using pairwise matching, shows long range tissue organization and cell coverage over an ˜9 mm2 region. (ii.) High magnification inset showing a high density of cell nuclei. (C) Schematic depicting the transverse cross sectioning of a ventricle construct. (D) Photograph of a transverse ventricle cross-section, fixed and suspended in phosphate buffered saline. (E) Immunofluorescent micrograph of the construct's cross section, showing a thin layer of cells covering the exterior of the ventricle.

### DETAILED DESCRIPTION

In the following description, it is understood that terms such as “top,” “bottom,” “middle,” “outward,” “inward,” and the like are words of convenience and are not to be construed as limiting terms. Reference will now be made in detail to embodiments of the disclosure, which are illustrated in the accompanying figures and examples. Referring to the drawings in general, it will be understood that the illustrations are for the purpose of describing particular embodiments of the disclosure and are not intended to limit the same.

Whenever a particular embodiment of the disclosure is said to comprise or consist of at least one element of a group and combinations thereof, it is understood that the embodiment may comprise or consist of any of the elements of the group, either individually or in combination with any of the other elements of that group.

As used herein, the terms “polymer fiber” and “polymeric fiber” refer to a fiber comprising a polymer. The fiber may also include some non-polymer components.

As used herein, a micron or nanometer dimension fiber or a fiber having a micron-scale or a nanometer-scale diameter refers to a fiber having a diameter of less than about 10 μm.

These, and other, aspects of the invention will be better appreciated and understood when considered in conjunction with the following description and the accompanying drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

Some embodiments described herein include methods for evaluating a functional response or functional activity (e.g., a biomechanical activity such as contraction or dilation, or an electrophysiological activity such action potential duration or conduction velocity) of an engineered three-dimensional tissue including a cavity or a lumen (e.g., a cardiac ventricle or a vascular lumen). Some embodiments described herein may show improved accuracy and/or improved sensitivity for evaluating a functional activity of engineered three-dimensional tissue including a cavity or a lumen. Some embodiments herein may more accurately model disease conditions affecting functional activity of an engineered three-dimensional tissue including a cavity or a lumen (e.g., a cardiac disease condition affecting an ejection fraction for an engineered ventricle).

Some embodiments described herein include methods for identifying a compound that modulates tissue function in three-dimensional tissue defining a cavity or a lumen. Some embodiments described herein include methods for identifying a compound that is useful for treating or preventing a disease affecting tissue function in three-dimensional tissue defining a cavity or a lumen. Some embodiments described herein provide in vitro methods that may be used determining the effect of a test compound on biological relevant parameters in order to enhance and speed-up the drug discovery and development process.

Some embodiments described herein include methods for evaluating a functional response or functional activity of an engineered three-dimensional tissue including a three-dimensional tissue scaffold defining a lumen or a cavity, where the three-dimensional tissue scaffold includes one or more micron-scale or nanometer-scale diameter polymeric fibers encircling the lumen or cavity at a helical angle with respect to a longitudinal axis of the lumen or cavity, at an azimuthal orientation with respect to the longitudinal axis of the lumen or cavity, or both. Some embodiments described herein include methods for identifying a compound that modulates tissue function using an engineered three-dimensional tissue.

Some embodiments described herein include methods for identifying a compound that modulates tissue function that employ an engineered three-dimensional tissue including a three-dimensional tissue scaffold defining a lumen or a cavity, where the three-dimensional tissue scaffold includes one or more micron-scale or nanometer-scale diameter polymeric fibers encircling the lumen or cavity at a helical angle with respect to a longitudinal axis of the lumen or cavity, at an azimuthal orientation with respect to the longitudinal axis of the lumen or cavity, or both. Some embodiments described herein include methods for identifying a compound that modulates tissue function using an engineered three-dimensional tissue.

Some embodiments described herein include methods for identifying a compound that is useful for treating or preventing a disease affecting tissue function that employ an engineered three-dimensional tissue including a three-dimensional tissue scaffold defining a lumen or a cavity, where the three-dimensional tissue scaffold includes one or more micron-scale or nanometer-scale diameter polymeric fibers encircling the lumen or cavity at a helical angle with respect to a longitudinal axis of the lumen or cavity, at an azimuthal orientation with respect to the longitudinal axis of the lumen or cavity, or both. Some embodiments described herein include methods for identifying a compound that modulates tissue function using an engineered three-dimensional tissue.

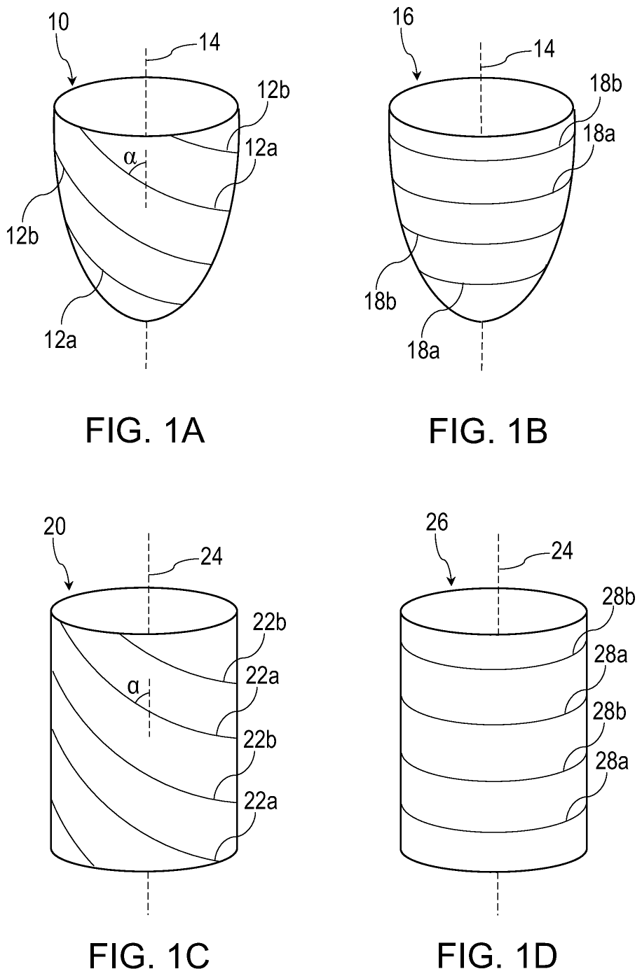

In some embodiments, methods include providing or obtaining a three-dimensional tissue scaffold defining a lumen or a cavity, the tissue scaffold including one or more polymeric fibers each having a micron-scale or nanometer-scale diameter, at least some of the one or more polymeric fibers encircling the lumen or cavity at a helical angle with respect to a longitudinal axis of the lumen or cavity, at an azimuthal orientation with respect to the longitudinal axis of the lumen or cavity, or both. FIG. 1A schematically depicts a three-dimensional tissue scaffold 10 comprising polymeric fibers 12a, 12b defining a cavity. The polymeric fibers 12a, 12b, encircle the cavity at a helical angle α with respect to a longitudinal axis 14 of the cavity. FIG. 1B schematically depicts a three-dimensional tissue scaffold 16 comprising polymeric fibers 18a, 18b defining a cavity where the polymeric fibers 16a, 16b encircle the cavity with an azimuthal or cylindrical orientation.

FIG. 1C schematically depicts a three-dimensional tissue scaffold 20 including polymeric fibers 22a, 22b defining a lumen. The polymeric fibers 22a, 22b encircle the cavity at a helical angle α with respect to a longitudinal axis 24 of the lumen. FIG. 1D schematically depicts a three-dimensional tissue scaffold 26 comprising polymeric fibers 28a, 28b defining a lumen where the polymeric fibers 28a, 28b encircle the lumen at with an azimuthal or cylindrical orientation with respect to the longitudinal axis 24 of the lumen.

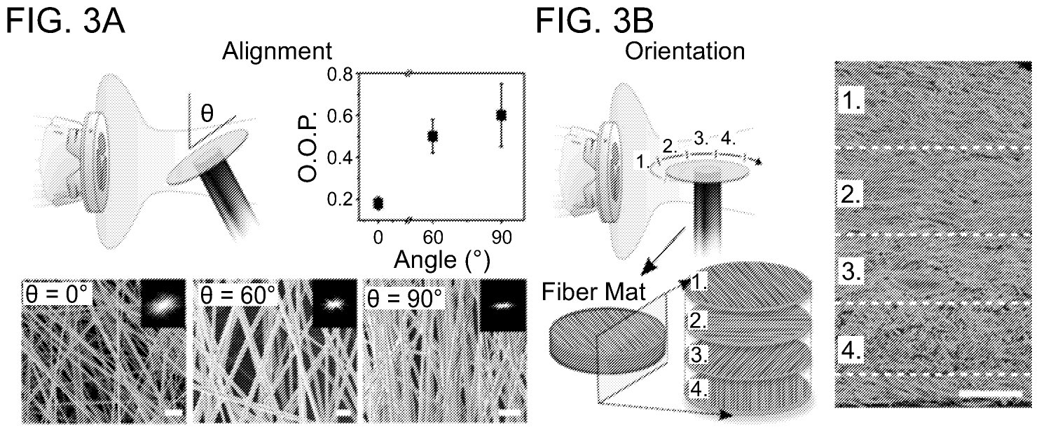

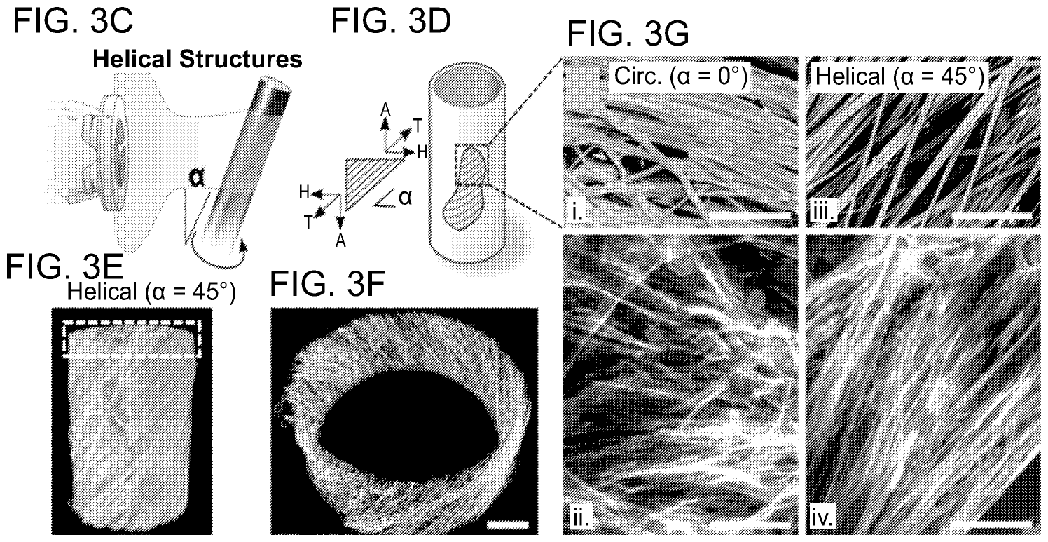

In each of FIGS. 1A, 1B, 1C and 1D, only two polymeric fibers are depicted for clarity and simplicity. See FIG. 3E for an image of an example three-dimensional tissue scaffold.

In some embodiments, the helical angle falls in a range of about 200 to about 70°. In some embodiments, the helical angle in a range of about 30° to about 60°. In some embodiments, the helical angle in a range of about 52° to about 58°. In some embodiments, the helical angle is in a range of 53.7° to 55.7°. In some embodiments, the helical angle falls a range of about 32.5° to about 35.8°. In some embodiments, the helical angle falls in a range of 34.3 to 36.3.

In some embodiments, the three-dimensional tissue scaffold includes: a first layer in which a first portion of the one or more polymeric fibers encircle the longitudinal axis at a first helical angle with respect the longitudinal axis of the lumen or cavity; and a second layer in which a second portion of the one or more polymeric fibers encircle the longitudinal axis at a second helical angle with respect to the longitudinal axis of the lumen or cavity that is different than the first helical angle. In some embodiments, a difference between the first helical angle and the second helical angle falls in a range of 20° to 70°. In some embodiments, the difference between the first helical angle and the second helical angle falls in a range of 30° to 60°. In some embodiments, the difference between the first helical angle and the second helical angle falls in a range of 52° to 58°. In some embodiments, the difference between the first helical angle and the second helical angle falls in a range of 32.5° to 35.8°

In some embodiments, the three-dimensional tissue scaffold includes: a first layer in which a first portion of the one or more polymeric fibers encircle the longitudinal axis at a first helical angle with respect the longitudinal axis of the lumen or cavity; and a second layer in which a second portion of the one or more polymeric fibers encircle the longitudinal axis at a second helical angle with respect to the longitudinal axis of the lumen or cavity that is different than the first helical angle. In some embodiments, a difference between the first helical angle and the second helical angle falls in a range of 20° to 70°. In some embodiments, the difference between the first helical angle and the second helical angle falls in a range of 30° to 60°. In some embodiments, the difference between the first helical angle and the second helical angle falls in a range of 52° to 580 or a range of 32.5° to 35.8°

As noted above, each of the polymeric fibers has a micron-scale or a nanometer-scale diameter (i.e., a diameter of the fiber is less than about 10 μm). In some embodiments, each fiber has diameter falling in a range of 0.5 μm to 10 μm. In some embodiments, each diameter has a fiber falling in a range of 0.5 μm to 5 μm.

In some embodiments, the polymeric fibers include gelatin. In some embodiments, the polymer fibers include one or more of polyurethane, polycaprolactone, nylon, poly(L-lactide-co-ε-caprolactone), extracellular matrix proteins (e.g. fibronectin, hyaluronic acid, laminin), deoxyribonucleic acid, collagen, elastin, starch, alginate, cellulose acetate, polyethylene glycol, polyethylene oxide, polylactic acid, pullulan, Polyhydroxyalkanoates, Polyethylene, polyacrylic acid, polyacrylamide, polyvinyl acetate, polyvinyl alcohol, zein, silk, keratine, polyvinyl chloride, Poly(4-Hydroxybutyrate), and poly(glycerol sebacate).

In some embodiments, the three-dimensional tissue scaffold also includes one or more of extracellular matrix proteins (e.g. fibronectin, hyaluronic acid, laminin), growth factors (e.g. endothelial growth factor, fetal bovine serum), cytokines, hormones (e.g. estrogen, testosterone, phytoestrogens), nanomaterials (e.g. carbon nanotubes, cellulose nanomaterials, gold, silver and cerium dioxide nanoparticles), and small molecule drugs.

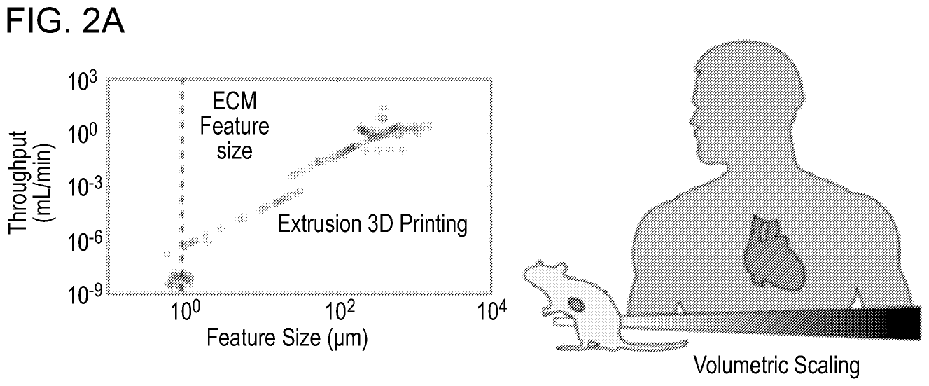

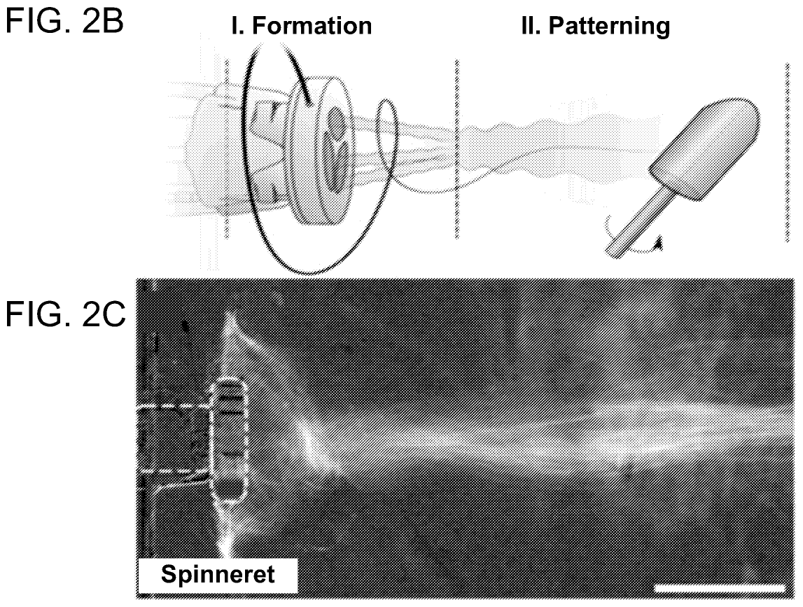

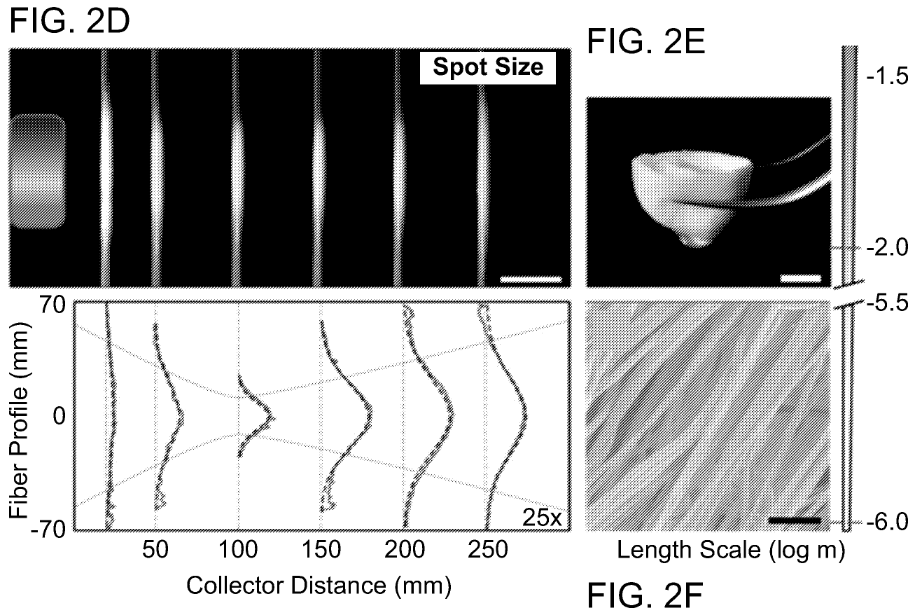

In some embodiments, providing or obtaining the three-dimensional scaffold includes rotating a reservoir holding a material comprising a polymer about a rotation axis to eject at least one jet of material from at least one orifice defined by an outer sidewall of the reservoir and directing at least one flow of gas through a portion of the reservoir radially inward of the outer sidewall. The at least one flow of gas is directed from an upstream first end of the reservoir past a downstream second end of the reservoir during rotation of the reservoir and ejection of the at least one jet of the material to form at least one micron or nanometer dimension polymeric fiber. The at least one flow of gas entrains the at least one micron or nanometer dimension polymeric fiber and forms a focused fiber deposition stream of the at least one micron or nanometer dimension polymeric fiber in a first direction, the first direction having an orientation parallel to the rotation axis of the reservoir or within 45 degrees of the rotation axis of the reservoir. The focused fiber deposition stream is collected on a target surface that is being rotated about a second rotation axis or is stationary, the target surface having a shape corresponding to at least a portion of the three-dimensional tissue scaffold (see, e.g., FIGS. 2B-2E, 3A, 3C, 6A-6F, 15A and accompanying description in the Example). In some embodiments, providing or obtaining the three-dimensional tissue scaffold comprises also includes changing the rotation axis of the target surface to a third rotation axis different than the second rotation axis and collecting the focused fiber deposition stream on the target surface when the target surface is being rotated about the third rotation axis (see, e.g., FIG. 3B and accompanying description in the Example). In some embodiments, an angle between the second rotation axis and the third rotation axis is 90° or less. In some embodiments, an angle between the second rotation axis and the third rotation axis falls in a range of 20° to 70°. Further information regarding formation of scaffolds via focused rotary jet spinning can be found in International Publication Number WO2020/150207 A1, which is incorporated herein in its entirety.

In some embodiments, methods further include providing or obtaining cells growing on or in the three-dimensional tissue scaffold to form a three-dimensional tissue defining the lumen or the cavity. Further description of growing cells on or in the three-dimensional tissue scaffold to form a three-dimensional tissue appears below in the section entitled “Culturing”.

In some embodiments, the cells include muscle cells. In some embodiments, the cells include cardiac muscle cells. In some embodiments, the cells include vascular epithelial cells. In some embodiments, the cells include at least one of vascular smooth muscle cells, cardiac myocytes, skeletal muscle cells, uterine smooth muscle cells, intestinal smooth muscle cells, myofibroblasts, airway smooth muscle cells, osteoblasts, myoblasts, neuroblasts, fibroblasts, glioblasts, germ cells, trophoblasts, lymphocytes, intestinal crypt cells, intestinal villi cells, chondrocytes, keratinocytes, connective tissue cells, glial cells, epithelial cells, endothelial cells, vascular endothelial cells, hormone-secreting cells, neural cells, and cells that will differentiate into muscle cells. Further description of types of cells that may be grown in or on the three-dimensional tissue scaffold also appears below in the section entitled “Culturing”. Further description and explanation of additional materials, components, agents that may be included or incorporated into the three-dimensional scaffold and/or a culture medium appears below in the section entitled “Culturing”.

In some embodiments, the three-dimensional tissue structure may include or be an engineered tissue ventricle. In some embodiments, the three-dimensional tissue structure may include or be an engineered heart. In some embodiments, the three-dimensional tissue structure may include or be a blood vessel. In some embodiments, the three-dimensional tissue structure may include or be an artery. In some embodiments, the three-dimensional tissue structure may include or be one or more of an artery, a blood vessel, a lymph node, a lymphatic vessel, an intestine, and an inner layer of a tongue.

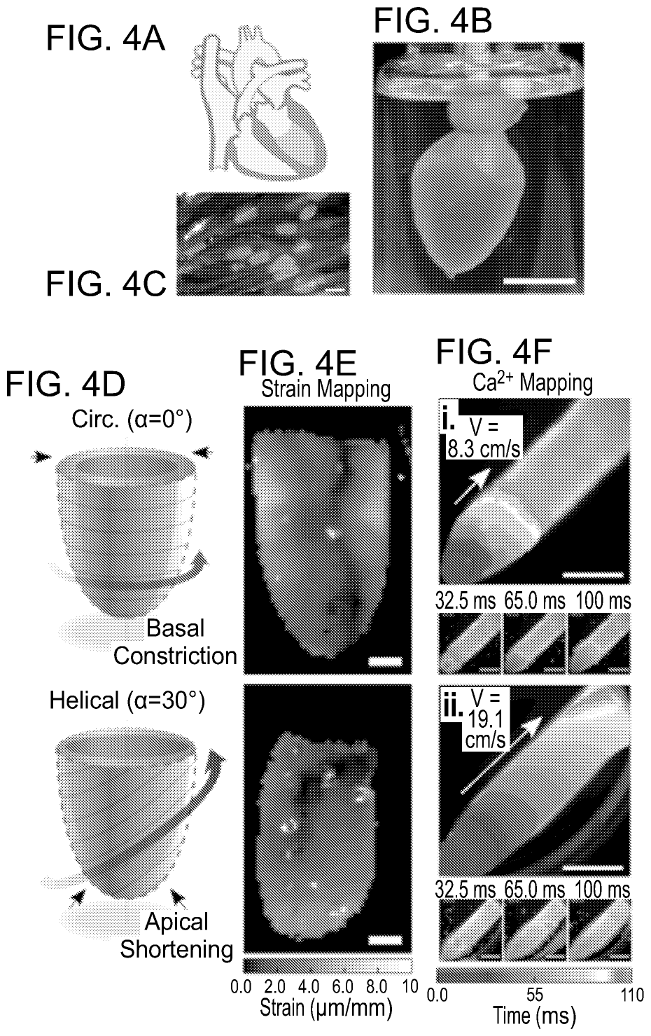

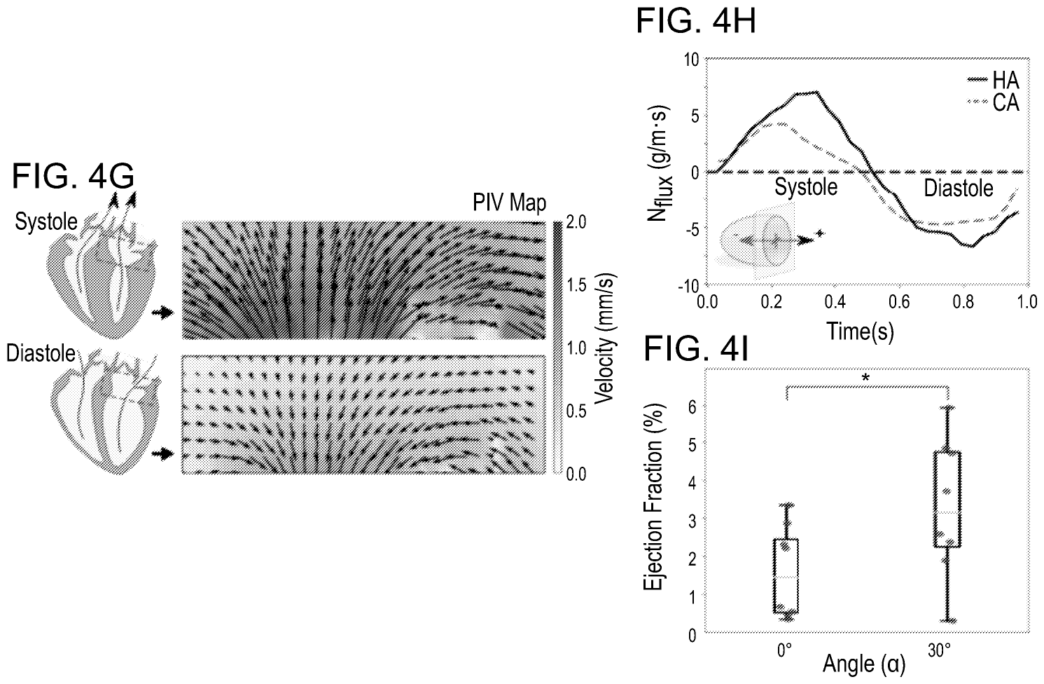

In some embodiments, the method also includes affixing a first portion of the three-dimensional tissue to a support where the cells are grown on or in the three-dimensional tissue scaffold to form the three-dimensional tissue before the three-dimensional tissue is affixed to the support (see, e.g., FIG. 4B and accompanying description in the Example). In some embodiments, the method also includes affixing a first portion of the three-dimensional tissue scaffold to a support where the cells are grown on or in the three-dimensional tissue scaffold to form the three-dimensional tissue after the three-dimensional tissue scaffold is affixed to the support. In some embodiments, the first portion affixed to the support is a first end of the three-dimensional tissue or the three-dimensional tissue scaffold. In some embodiments, the first portion affixed to the support is not disposed at an end of the three-dimensional tissue or of the three-dimensional tissue scaffold.

In some embodiments, the affixing is mechanical, e.g., via a clamp, sutures, a staple, a puncture, thermal based fusion, annealing and welding, knitting, compression welding, magnetic adhesion, or a combination of any of the aforementioned. In some embodiments, the affixing may be through use of an adhesive, a sealant, or a glue, via chemical fixation and crosslinking, chemical annealing, electrostatic bonding, cold-fusion welding, or plasma coating, or a combination of any of the aforementioned.

In some embodiments, the method also includes measuring rotational displacement of at least a portion of the three-dimensional tissue relative to the support over a period of time caused by functional activity of three-dimensional tissue. In some embodiments, the method also includes measuring strain of at least a portion of the three-dimensional tissue relative to the support over a period of time caused by functional activity of the three-dimensional tissue.

In some embodiments, the functional activity is a biomechanical activity. In some embodiments, the biomechanical activity is contractility (see, e.g., FIGS. 4D-4E, 7a-7d, 15 and accompany description in the Example). In some embodiments, the biomechanical activity is one or more of contractility, cell stress, cell swelling, and rigidity. In some embodiments, the biomechanical activity is one or more of stem cell activation, stem cell maturation, tissue morphogenesis, and tissue remodeling.

In some embodiments the functional activity is an electrophysiological activity. In some embodiments, the electrophysiological activity is or is related to a voltage parameter. In some embodiments, the electrophysiological activity is, includes, or is related to a voltage parameter selected from the group consisting of action potential, action potential duration (APD), conduction velocity (CV), refractory period, wavelength, restitution, bradycardia, tachycardia, reentrant arrhythmia, and a calcium flux parameter. In some embodiments, the calcium flux parameter comprises one or more of intracellular calcium transient, transient amplitude, rise time (contraction), decay time (relaxation), total area under the transient (force), restitution, focal, and spontaneous calcium release.

In some embodiments, the functional activity is both a biomechanical activity and an electrophysiological activity.

In some embodiments, the functional activity is initiated by a stimulus. In some embodiments, the functional activity is preceded by a stimulus. In some embodiments, the functional activity is in response to a stimulus. In some embodiments, the functional activity is modified by a stimulus.

In some embodiments, the method is a method of evaluating the functional activity of the three-dimensional tissue. In some embodiments the method also includes stimulating the three-dimensional tissue before, during, or before and during obtaining the images over the period of time, to initiate the functional activity of the three-dimensional tissue, or to affect the functional activity of the three-dimensional tissue. In some embodiments, the method is a method of evaluating the functional activity of the three-dimensional tissue in response to the stimulation.

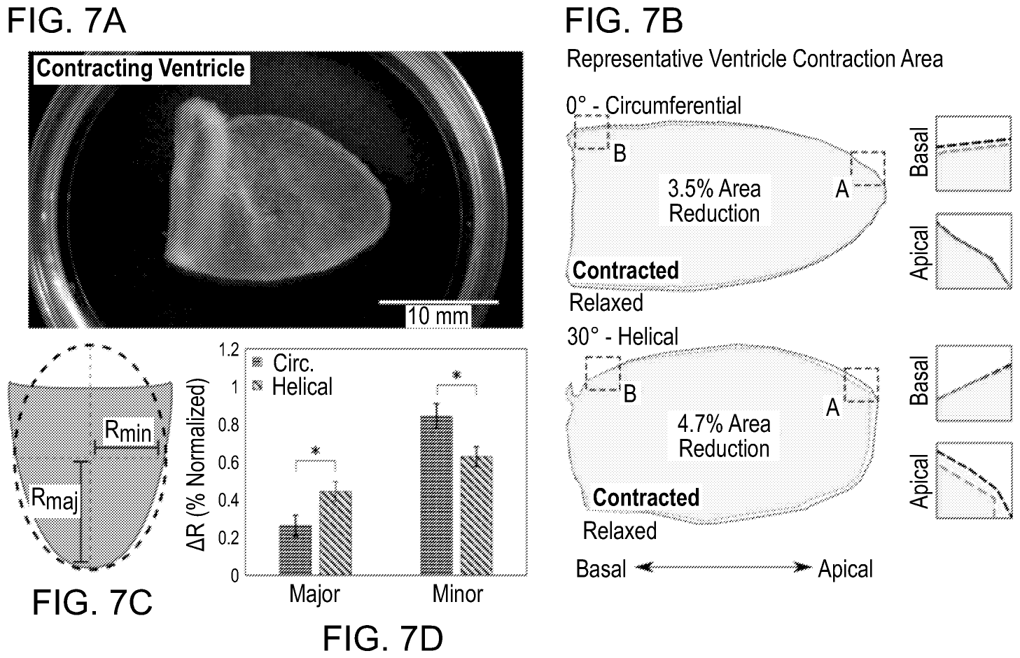

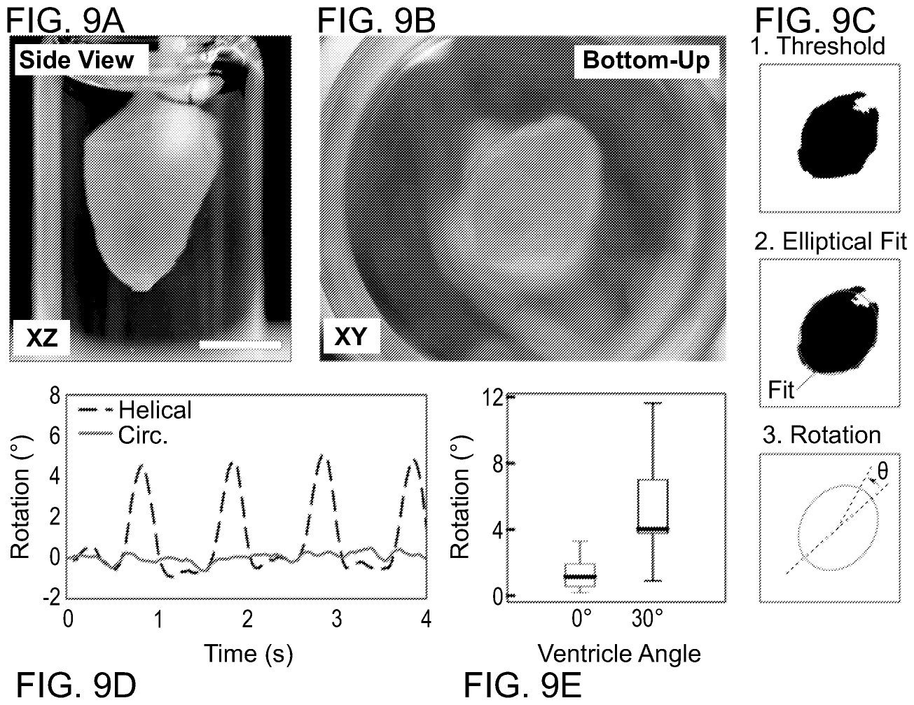

Measuring rotational displacement or measuring strain may be achieved using any suitable method. In some embodiments measuring rotational displacement or measuring strain of at least the second portion of the three-dimensional tissue relative to the support over the period of time includes obtaining images of at least the portion of the three dimensional-tissue over the period of time. Any suitable imaging technique may be employed. In some embodiments, images of at least the portion of the three-dimensional tissue include images of a two-dimensional perimeter or edge of the three-dimensional tissue (e.g., an outer edge of the three-dimensional tissue) (see, e.g., FIGS. 7a-7c, 9A-9E and accompanying description in the Example). In some embodiments, images of at least the second portion of the three-dimensional disuse may be processed using any suitable filters or for edge detection to locate a two-dimensional perimeter or edge of the three-dimensional tissue. In some embodiments, a change is measured in an orientation of the two-dimensional perimeter or edge of the three-dimensional over the period of time (see, e.g., FIGS. 9A-9E and accompanying description in the Example). In some embodiments, the two-dimensional perimeter or edge of the three-dimensional tissue may be fitted to one or more shapes to determine an orientation of the two-dimensional perimeter (see, e.g., FIGS. 7a-7c, 9A-9E and accompanying description in the Example).

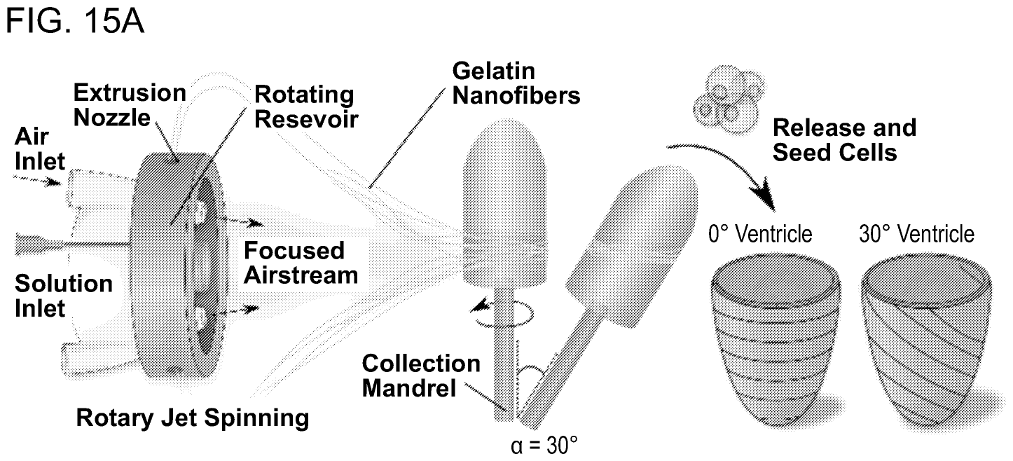

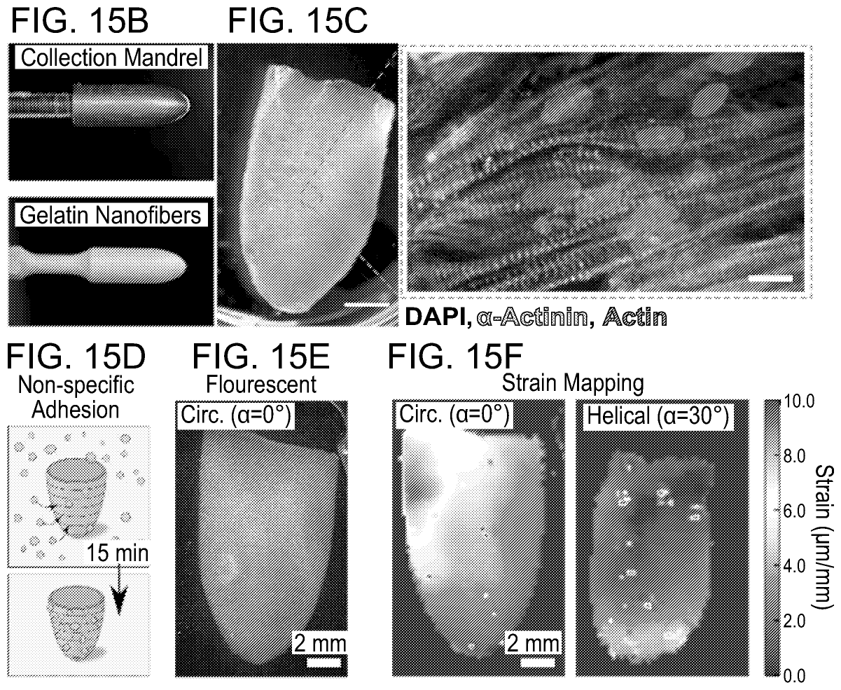

In some embodiments, the three-dimensional tissue or the three-dimensional tissue scaffold may be exposed to a solution including fiducial markers (e.g., optical fiducial markers) such that the fiducial markers adhere to or are incorporated into the three-dimensional tissue or the three dimensional tissue scaffold (see, e.g., FIGS. 15d-15e and accompanying description in the Example). In some embodiments, fiducial markers (e.g., optical fiducial markers) are incorporated into the three-dimensional tissue scaffold during formation of the three-dimensional tissue scaffold. In some embodiments, measuring rotational displacement of at least the portion of the three-dimensional tissue or measuring strain of at least the portion of the three-dimensional tissue over time includes performing imaging of the fiducial markers in or on at least the portion of the three-dimensional tissue over the period of time (see, e.g., FIGS. 4D, 15d-15f and accompanying description in the Example).

In some embodiments, the fiducial markers are optical fiducial markers. In some embodiments, the optical fiducial markers are or include fluorescent beads. In some embodiments, the fluorescent beads adhere to the three-dimensional tissue scaffold (see, e.g., FIGS. 15d-15e and accompanying description in the Example).

In some embodiments, the fiducial markers include one or more of reflective beads, reflective particles, optically scattering beads, optically scattering particles (e.g., such as could be detected through darkfield scattering). In some embodiments, the imaging is, at least in part, darkfield imaging.

In some embodiments, the optical fiducial markers may include a fluorescent dye or stain (e.g. an immunofluorescent dye or stain). For example, the nuclei of the cells may be stained, e.g., using a fluorescent dye, e.g., DAPI.

Imaging of the fiducial markers may be via any suitable imaging method. For example, imaging of optical fiducial markers may be imaging in the visible spectrum, fluorescence imaging, dark field scattering, laser scattering, via microscopy, via confocal microcopy, using a macroscope, or using any other suitable technique. In some embodiments, imaging of fiducial markers may be via ultrasound, using electromagnetic wavelengths in the non-visible spectrum, or via any other suitable imaging method.

In some embodiments, one or more strain sensors may be attached to or incorporated into the three-dimensional tissue, and measuring rotational displacement of at least the second portion of the three-dimensional tissue relative to a support over the period of time or measuring strain of at least the second portion of the three-dimensional tissue relative to the support over the period of time is via the one or more strain sensors. In some embodiments, the one or more strain sensors are electronic strain sensors. In some embodiments, the one or more strain sensors are piezoelectric strain sensors. In some embodiments, the one or more strain sensors are conductance change strain sensors. In some embodiments, the one or more strain sensors include one or more of an electronic strain sensor, a piezoelectric strain sensor, and a conductance change strain sensor.

In some embodiments, measuring strain of at least the second portion of the three-dimensional tissue relative to the support over the period of time includes performing strain mapping of at least the portion of the three dimensional-tissue over the period of time. The strain mapping may involve any suitable method. In some embodiments, the strain mapping may be via tracking of fiducial markers in the second portion of the three-dimensional tissue (see, e.g., FIGS. 4D, 15d-15f and accompanying description in the Example). In some embodiments, the method may further include performing particle imaging velocimetry (PIV) using the fiducial markers. In some embodiments, the strain mapping may be via multiple electronic strain sensors attached to or incorporated into the three-dimensional tissue.

In some embodiments, measuring rotational displacement or measuring strain of at least the second portion of the three-dimensional tissue relative to the support over the period of time includes measuring over at least one cycle of a cyclic functional activity. In some embodiments, measuring rotational displacement or measuring strain of at least the second portion of the three-dimensional tissue relative to the support over the period of time includes measuring multiple cycles of a cyclic functional activity (see, e.g., FIG. 9D and accompanying description in the Example).

In another embodiment, a method includes providing or obtaining a three-dimensional tissue scaffold defining a lumen or a cavity. The tissue scaffold includes one or more polymeric fibers each having a micron-scale or nanometer-scale diameter. At least some of the one or more polymeric fibers encircle the lumen or cavity at a helical angle with respect to a longitudinal axis of the lumen or cavity, at an azimuthal orientation with respect to the longitudinal axis of the lumen or cavity, or both. The method also includes providing or obtaining cells growing on or in the three-dimensional tissue scaffold to form a three-dimensional tissue defining the lumen or the cavity. The cells may be any suitable cells described herein.

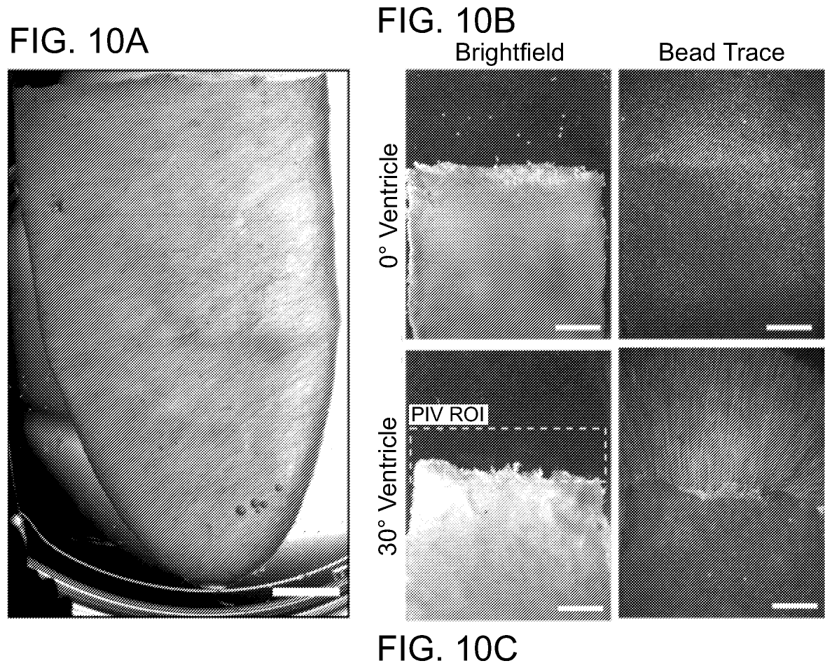

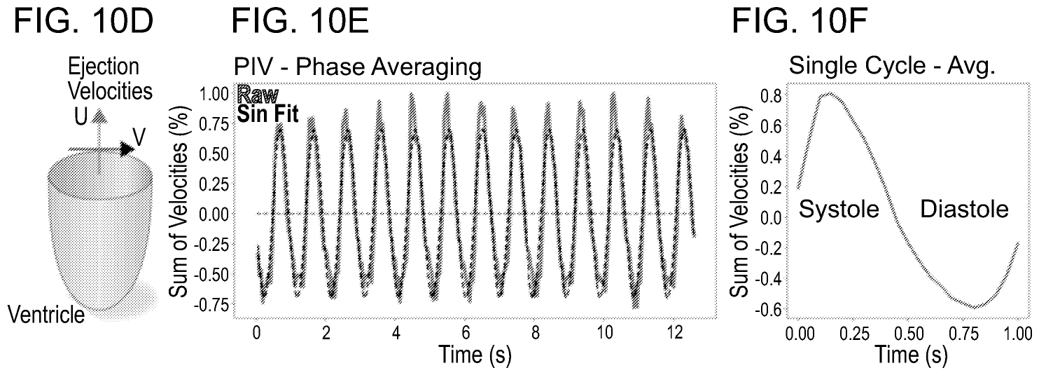

The method also includes exposing the three-dimensional tissue to a medium including fiducial markers. In some embodiments, the fiducial markers include or are attached to beads or particles. In some embodiments, the beads or particles are neutrally buoyant in the medium. In some embodiments, the beads or particles are fluorescent (see, e.g., FIGS. 4G, 10A-10C and accompanying description in Example) In some embodiments, the beads or particles are reflective or scattering. In some embodiments, the imaging, is, at least in part, darkfield imaging.

In some embodiments, the method also includes obtaining images of an area including or adjacent to an opening of the lumen or cavity over a period of time (see, e.g., FIGS. 10B-10C and accompanying description in the Example). In some embodiments, the method also includes performing particle imaging velocimetry on fiducial markers in the images of the area including or adjacent to the opening of the lumen or cavity (see, e.g., FIGS. 4G, 15d-15f and accompanying description in the Example). Any suitable software, program, app or algorithm may be employed for performing particle imaging velocimetry (e.g., OpenPIV/python open source software). In some embodiments, the method also includes determining a volume, mass flux, or velocity of fluid flow out of or into the opening due to or affected by a functional activity of the three-dimensional tissue based on the particle imaging velocimetry (see, e.g., FIGS. 10D-F and accompanying description in the Example).

In some embodiments, the cells include muscle cells. In some embodiments, the cells include cardiac muscle cells. In some embodiments, the cells include vascular epithelial cells. In some embodiments, the cells include at least one of vascular smooth muscle cells, cardiac myocytes, skeletal muscle cells, uterine smooth muscle cells, intestinal smooth muscle cells, myofibroblasts, airway smooth muscle cells, osteoblasts, myoblasts, neuroblasts, fibroblasts, glioblasts, germ cells, trophoblasts, lymphocytes, intestinal crypt cells, intestinal villi cells, chondrocytes, keratinocytes, connective tissue cells, glial cells, epithelial cells, endothelial cells, vascular endothelial cells, hormone-secreting cells, neural cells, and cells that will differentiate into muscle cells.

In some embodiments, the method is a method of evaluating the functional activity of the three-dimensional tissue. In some embodiments the method also includes stimulating the three-dimensional tissue before, during, or before and during obtaining the images over the period of time, to initiate the functional activity of the three-dimensional tissue, or to affect the functional activity of the three-dimensional tissue. In some embodiments, the method is a method of evaluating the functional activity of the three-dimensional tissue in response to the stimulation.

The methods of the invention are also useful for measuring tissue activities or functions, and investigating tissue developmental biology and disease pathology, as well as in drug discovery and toxicity testing.

Accordingly, in one aspect, the present invention provides methods for identifying a compound that modulates a tissue function.

In some embodiments of the methods for identifying a compound useful for treating or preventing a disease affecting tissue function, the methods include contacting the three-dimensional tissue structure with a test compound. In some embodiments, measuring rotational displacement of at least the second portion of the three-dimensional tissue with respect to the support over a period of time includes: measuring a first rotational displacement of at least the second portion of the three-dimensional tissue relative to the support in the presence of the test compound over a first period of time; and measuring a second rotational displacement at least the second portion of the three-dimensional tissue relative to the support in the absence of the test compound over a second period of time. The method also includes comparing the first rotational displacement with the second rotational displacement. A modulation of the first rotational displacement in the presence of the test compound as compared to the second rotational displacement in the absence of the test compound indicates that the test compound modulates tissue function, thereby identifying a compound that modulates tissue function.

In some embodiments, the first rotational displacement and/or second rotational displacement are based on measurements of one or more cycles of a cyclic functional activity. In some embodiments, the first rotational displacement and/or second rotational displacement is based on measurement over multiple cycles (e.g., an average per cycle, a median per cycle, an average of a peak per cycle, etc.).

In some embodiments of the methods for identifying a compound useful for treating or preventing a disease affecting tissue function, the methods include contacting the three-dimensional tissue structure with a test compound, and measuring strain of at least the second portion of the three-dimensional tissue relative to the support over the period of time includes: measuring a first strain of at least the second portion of the three-dimensional tissue relative to the support in the presence of the test compound over a first period of time; and measuring a second strain of at least the second portion of the three-dimensional tissue relative to the support in the absence of the test compound over a second period of time. The method also include comparing the first strain with the second strain. A modulation of the first strain in the presence of the test compound as compared to the second strain in the absence of the test compound indicates that the test compound modulates tissue function, thereby identifying a compound that modulates tissue function

In some embodiments, the first strain and/or second strain are based on measurements of one or more cycles of a cyclic functional activity. In some embodiments, the first strain and/or second strain is based on measurement over multiple cycles (e.g., an average or a median per cycle). In some embodiments, the first strain and/or second strain are based on strain values determined from strain mapping.

In one embodiment, the three-dimensional tissue structure is a functional three-dimensional muscle structure, e.g. functional three-dimensional tissue cardiac tissue structure, and a compound that increases rotational displacement in the presence of the test compound as compared to the absence of the test compound is identified as a compound that increases a muscle tissue function, e.g., a biomechanical and or electrophysiological tissue function. For example, a test compound that increases rotational displacement of a functional muscle tissue structure may be identified as a compound that increases muscle tissue contractility.

In another embodiment, the three-dimensional tissue structure is a functional three-dimensional vascular smooth muscle cell structure, e.g. functional three-dimensional blood vessel tissue structure, and a compound that increases strain in the presence of the test compound as compared to the absence of the test compound is identified as a compound that increases a vascular smooth muscle cell function, e.g., a biomechanical and or electrophysiological tissue function. For example, a test compound that increases strain of a functional vascular smooth muscle cell structure may be identified as a compound that increases blood flow.

In another embodiment, the screening methods of the invention include contacting the three-dimensional tissue structure with a test compound and obtaining images of the area including or adjacent to the opening of the lumen or cavity over a period of time include obtaining first images of the area in the presence of the test compound over a first period of time; and obtaining second images of the area in the absence of the test compound over a second period of time. Determining a volume, mass flux, or velocity of fluid flow out of or into the opening due to or affected by a functional activity of the three-dimensional tissue based on the particle imaging velocimetry includes determining a first volume, mass flux, or velocity of fluid flow out of or into the opening during the first period of time; and determining a second volume, mass flux, or velocity of fluid flow out of or into the opening during the second period of time; and comparing the first volume, mass flux, or velocity of fluid flow and the second volume, mass flux, or velocity of fluid flow. A modulation of the first volume, mass flux, or velocity of fluid flow as compared to the second volume, mass flux, or velocity of fluid flow indicates that the test compound modulates the tissue function.

In some embodiments, the first volume, mass flux, or velocity of fluid flow and/or the second first volume, mass flux, or velocity of fluid flow are based on measurements of one or more cycles of a cyclic functional activity. In some embodiments, the first volume, mass flux, or velocity of fluid flow and/or the second first volume, mass flux, or velocity of fluid flow (e.g., an average per cycle, a median per cycle, an average of a peak per cycle, etc.). In some embodiments, the first strain and/or second strain are based on strain values determined from strain mapping.

In one embodiment, the three-dimensional tissue structure is a functional three-dimensional muscle structure, e.g. functional three-dimensional tissue cardiac tissue structure, and a compound that increases volume, mass flux, or velocity of fluid flow in the presence of the test compound as compared to the absence of the test compound is identified as a compound that increases a muscle tissue function, e.g., a biomechanical and or electrophysiological tissue function. For example, a test compound that increases volume, mass flux, or velocity of fluid flow of a functional muscle tissue structure may be identified as a compound that increases muscle tissue contractility.

In one embodiment, the three-dimensional tissue structure is a functional three-dimensional lymphatic vessel structure, and the method is a method of evaluating the functional activity (e.g., the biomechanical function) of the lymphatic vessel structure.

In one embodiment, the three-dimensional tissue structure is a functional three-dimensional lymphatic vessel structure, and a compound that increases volume, mass flux, or velocity of fluid flow in the presence of the test compound as compared to the absence of the test compound is identified as a compound that increases a lymphatic tissue function, e.g., a biomechanical function. For example, a test compound that increases volume, mass flux, or velocity of fluid flow of the functional lymphatic vessel structure may be identified as a compound that increases lymph transport velocity. As another example, a test compound that decreases volume, mass flux, or velocity of fluid flow through the functional lymphatic vessel structure may be identified as a compound that decreases lymph transport velocity.

In one embodiment, the three-dimensional tissue structure is a three-dimensional intestinal tissue structure, and the method is a method of evaluating functional activity (e.g., biomechanical function) of the intestinal tissue structure.

In one embodiment, the three-dimensional tissue structure is a three-dimensional intestinal tissue structure, and a compound that increases volume, mass flux, or velocity of fluid flow in the presence of the test compound as compared to the absence of the test compound is identified as a compound that improves an intestinal tissue function. For example, a test compound that increases volume, mass flux, or velocity of fluid flow through the intestinal tissue structure may be identified as a compound that increases a transport or digestion velocity through the intestinal track.

In one embodiment, the three-dimensional tissue structure is a functional three-dimensional muscular hydrostat structure, and the method is a method of evaluating the functional activity (e.g., a biomechanical and/or electrophysiological tissue function) of the muscular hydrostat structure. For example, the three-dimensional muscular hydrostat structure may be a model of a tongue.

In one embodiment, the three-dimensional tissue structure is a functional three-dimensional muscular hydrostat structure, and a compound that increases volume, mass flux, or velocity of fluid flow in the presence of the test compound as compared to the absence of the test compound is identified as a compound that increases a muscular hydrostat tissue function (e.g., a biomechanical and/or electrophysiological tissue function).

In one embodiment, the three-dimensional tissue structure is a functional three-dimensional uterine, e.g., myometrium, structure, and the method is a method of evaluating the functional activity (e.g., a biomechanical and/or electrophysiological tissue function) of the uterine structure. For example, the three-dimensional uterine, e.g., myometrium, structure, may be a model of a uterus.

In another aspect, the present invention provides methods for identifying a compound useful for treating or preventing a disease affecting tissue function. In some embodiments, the disease is a muscular disease, pericarditis, endocarditis, cardiac fibrosis, or an arterial disease. In some embodiments the muscular disease is hypertension structural arrhythmia, ischemia, an inherited heart condition (e.g. Barth syndrome, familial dilated cardiomyopathy, or catecholaminergic polymorphic ventricular tachycardia), pulmonary valve regurgitation, or mitral valve regurgitation. In some embodiments, the disease is a vascular disease (e.g., pulmonary, coronary hypertension, artherosclerosis). In some embodiments, the disease is tumor formation and/or growth. For example, tumors can have a hydrostatic pressure in a central cavity due to necrotic tissues at the center. This hydrostatic pressure is important for therapeutic drug delivery, and the methods disclosed herein permit study of this.

In some embodiments of the methods for identifying a compound useful for treating or preventing a disease affecting tissue function, the methods include contacting the three-dimensional tissue structure with a test compound, and measuring rotational displacement of the at least a second portion of the three-dimensional tissue with respect to the support over a period of time includes measuring a first rotational displacement of the at least the second portion of the three-dimensional tissue relative to the support in the presence of the test compound over a first period of time; and measuring a second rotational displacement of the at least the second portion of the three-dimensional tissue relative to the support in the absence of the test compound over a second period time, The method also includes comparing the first rotational displacement with the second rotational displacement, where a modulation of the first rotational displacement in the presence of the test compound as compared to the second rotational displacement in the absence of the test compound indicates that the test compound modulates tissue function, thereby identifying a compound useful for treating or preventing a disease affecting tissue function.

In one embodiment, the three-dimensional tissue structure is a functional three-dimensional muscle structure, e.g. functional three-dimensional tissue cardiac tissue structure, and a compound that increases rotational displacement in the presence of the test compound as compared to the absence of the test compound is identified as a compound that increases a muscle tissue function, e.g., a biomechanical and or electrophysiological tissue function, and is useful for treating or preventing a disease affecting muscle tissue function. For example, a test compound that increases rotational displacement of a functional muscle tissue structure may be identified as a compound that increases muscle tissue contractility and is thus, useful for treating or preventing a disease affecting muscle tissue contractility, such as hypertension.

In some embodiments of the methods for identifying a compound useful for treating or preventing a disease affecting tissue function, the methods include contacting the three-dimensional tissue structure with a test compound, and measuring a strain of at least the second portion of the three-dimensional tissue relative to the support over a period of time includes measuring a first strain of at least the second portion of the three-dimensional tissue relative to the support in the presence of the test compound over a first period of time; and measuring a second strain of at least the second portion of the three-dimensional tissue relative to the support in the absence of the test compound over a second period of time. The method also includes comparing the first strain with the second strain, where a modulation of the first strain in the presence of the test compound as compared to the second strain in the absence of the test compound indicates that the test compound modulates tissue function, thereby identifying a compound useful for treating or preventing a disease affecting tissue function.

In one embodiment, the three-dimensional tissue structure is a functional three-dimensional muscle structure, e.g. functional three-dimensional tissue cardiac tissue structure, and a compound that increases strain in the presence of the test compound as compared to the absence of the test compound is identified as a compound that increases a muscle tissue function, e.g., a biomechanical and or electrophysiological tissue function, and is useful for treating or preventing a disease affecting muscle tissue function. For example, a test compound that increases strain of a functional muscle tissue structure may be identified as a compound that increases muscle tissue contractility and is thus, useful for treating or preventing a disease affecting muscle tissue contractility, such as hypertension.

In one embodiment, the three-dimensional tissue structure is a functional three-dimensional blood vessel tissue structure, e.g. a functional three-dimensional arterial vessel structure, and a compound that increases strain in the presence of the test compound as compared to the absence of the test compound is identified as a compound that increases a blood vessel function, e.g., a biomechanical function, and is useful for treating or preventing a disease affecting blood vessel function. For example, a test compound that increases strain of a blood vessel tissue structure may be identified as a compound that increases a blood vessel function and is thus, useful for treating or preventing a disease affecting blood vessels, such as arterial disease (e.g., hypertension).

In one embodiment, the three-dimensional tissue structure is a functional three-dimensional muscular hydrostat structure, and a compound that increases strain in the presence of the test compound as compared to the absence of the test compound is identified as a compound that increases a muscular hydrostat tissue function (e.g., a biomechanical and/or electrophysiological tissue function). For example, a test compound that increases strain of muscular hydrostat structure may be identified as a compound that increases a muscular hydrostat tissue function and is thus, useful for treating or preventing a disease affecting muscular hydrostats (e.g., the tongue), such as muscular dystrophy.

In some embodiments of the methods for identifying a compound useful for treating or preventing a disease affecting tissue function, the methods include contacting the three-dimensional tissue structure with a test compound, and obtaining images of the area including or adjacent to the opening of the lumen or cavity over a period of time includes: obtaining first images of the area in the presence of the test compound over a first period of time; and obtaining second images of the area in the absence of the test compound over a second period of time. Determining a volume, mass flux, or velocity of fluid flow out of or into the opening due to or affected by a functional activity of the three-dimensional tissue based on the particle imaging velocimetry includes determining a first volume, mass flux, or velocity of fluid flow out of or into the opening during the first period of time; and determining a second volume, mass flux, or velocity of fluid flow out of or into the opening during the second period of time. The method also includes comparing the first volume, mass flux, or velocity of fluid flow and the second volume, mass flux, or velocity of fluid flow, where a modulation of the first volume, mass flux, or velocity of fluid flow as compared to the second volume, mass flux, or velocity of fluid flow indicates that the test compound modulates tissue function, thereby identifying a compound useful for treating or preventing a disease affecting tissue function.

As used herein, the various forms of the term “modulate” are intended to include stimulation (e.g., increasing or upregulating a particular response or activity) and inhibition (e.g., decreasing or downregulating a particular response or activity).

As used herein, the term “contacting” (e.g., contacting a three-dimensional tissue structure with a test compound) is intended to include any form of interaction (e.g., direct or indirect interaction) of a test compound and a three-dimensional tissue structure. The term contacting includes incubating a compound and a three-dimensional tissue structure (e.g., adding the test compound to a three-dimensional tissue structure).

Test compounds, may be any agents including chemical agents (such as toxins), small molecules, pharmaceuticals, peptides, proteins (such as antibodies, cytokines, enzymes, and the like), and nucleic acids, including gene medicines and introduced genes, which may encode therapeutic agents, such as proteins, antisense agents (i.e., nucleic acids comprising a sequence complementary to a target RNA expressed in a target cell type, such as RNAi or siRNA), ribozymes, and the like. In some embodiments, a test compound is an endogenous factor, such as a hormone, a metabolic product, etc.), a diet supplement, and a pollutant, and other toxins.

The three-dimensional tissue structure may be contacted by the test compound by any suitable means. For example, the test compound may be added drop-wise onto the surface of a three-dimensional tissue structure of the invention and allowed to diffuse into or otherwise enter the three-dimensional tissue structure, or it can be added to the nutrient medium and allowed to diffuse through the medium. In the embodiment where the three-dimensional tissue structure is cultured in a multi-well plate, each of the culture wells may be contacted with a different test compound or the same test compound. In one embodiment, the screening platform includes a microfluidics handling system to deliver a test compound and simulate exposure of the microvasculature to drug delivery.

Numerous tissue functions and tissue activities, e.g., biomechanical and electrophysiological activities, can be evaluated using the methods of the invention.

Exemplary biomechanical tissue functions include one or more of contractility, cell stress, cell swelling, and rigidity; or one or more of stem cell activation, stem cell maturation, tissue morphogenesis, and tissue remodeling in some embodiments.

Exemplary electrophysiological tissue functions include a voltage parameter selected from the group consisting of action potential, action potential duration (APD), conduction velocity (CV), refractory period, wavelength, restitution, bradycardia, tachycardia, reentrant arrhythmia, and/or a calcium flux parameter, e.g., intracellular calcium transient, transient amplitude, rise time (contraction), decay time (relaxation), total area under the transient (force), restitution, focal and spontaneous calcium release; or one or more of intracellular calcium transient, transient amplitude, rise time (contraction), decay time (relaxation), total area under the transient (force), restitution, focal, and spontaneous calcium release in some embodiments.

In some embodiments, the methods of the invention may be used to determine the toxicity of a test compound by evaluating, e.g., the effect of the compound on an electrophysiological or biomechanical response of a three-dimensional tissue structure. For example, opening of calcium channels results in influx of calcium ions into the cell, which plays an important role in excitation-contraction coupling in cardiac and skeletal muscle fibers. The reversal potential for calcium is positive, so calcium current is almost always inward, resulting in an action potential plateau in many excitable cells. These channels are the target of therapeutic intervention, e.g., calcium channel blocker sub-type of anti-hypertensive drugs. Candidate drugs may be tested in the electrophysiological or biomechanical characterization assays described herein to identify those compounds that may potentially cause adverse clinical effects, e.g., unacceptable changes in cardiac excitation, that may lead to arrhythmia.

The bioengineered tissues and methods of the invention are also useful for evaluating the effects of particular delivery vehicles for therapeutic agents e.g., to compare the effects of the same agent administered via different delivery systems, or simply to assess whether a delivery vehicle itself (e.g., a viral vector or a liposome) is capable of affecting the biological activity of the three-dimensional tissue structure.

### Culturing

The three-dimensional tissue scaffolds of the invention may be seeded with a population of cells to fabricate the three-dimensional tissue structures of the invention.

Accordingly, in some embodiments, a three-dimensional tissue scaffold is seeded with a plurality cells and cultured in an incubator under physiologic conditions (e.g., at 37° C.) until the cells form a functional tissue structure.

A functional tissue structure is an in vitro three-dimensional tissue that recapitulates one or more three-dimensional interactions that occur between cells and their surrounding tissue in vivo. For example, the sarcomeres in the muscle cells and/or the cell themselves of a functional three-dimensional muscle tissue structure may be anisotropically aligned and/or the tissue is electrically functional and actively contractile.

Any appropriate cell culture method may be used. The seeding density of the cells will vary depending on the cell size and cell type, but can easily be determined by methods known in the art. In one embodiment, cells are seeded at a density of between about 1×10⁵ to about 6×10⁵ cells/cm², or at a density of about 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, or about 9.5×10⁶. Values and ranges intermediate to the above-recited values and ranges are also contemplated by the present invention.

In some embodiments, a three-dimensional tissue scaffold is contacted with living cells during the fabrication process such that a structure populated with cells or fibers surrounded (partially or totally) with cells are produced. The scaffold may also be contacted with additional agents, such as proteins, nucleotides, lipids, drugs, pharmaceutically active agents, biocidal and antimicrobial agents during the fabrication process such that polymeric fibers are produced which contain these agents. For example, fibers comprising living cells may be fabricated by providing a polymer and living cells in a solution of cell media at a concentration that maintains cell viability.

Suitable cells for use in the invention may be normal cells, abnormal cells (e.g., those derived from a diseased tissue, or those that are physically or genetically altered to achieve an abnormal or pathological phenotype or function), normal or diseased muscle cells, stem cells (e.g., embryonic stem cells), or induced pluripotent stem cells. Suitable cells include vascular smooth muscle cells, cardiac myocytes, skeletal muscle cells, uterine smooth muscle cells, intestinal smooth muscle cells, myofibroblasts, airway smooth muscle cells, osteoblasts, myoblasts, neuroblasts, fibroblasts, glioblasts, germ cells, hepatocytes, chondrocytes, keratinocytes, connective tissue cells, glial cells, epithelial cells, endothelial cells, vascular endothelial cells, hormone-secreting cells, neural cells, trophoblasts, intestinal crypt cells, intestinal villi cells, and cells that will differentiate into muscle cells. Such cells may be seeded on the scaffold and cultured to form a functional tissue, such as a three-dimensional tissue.

Cells for seeding can be cultured in vitro, derived from a natural source, genetically engineered, or produced by any other means. Any natural source of cells may be used.

The term “progenitor cell” is used herein to refer to cells that have a cellular phenotype that is more primitive (e.g., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “progenitor cell” is used herein synonymously with “stem cell.”

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation”.

The term “embryonic stem cell” is used to refer to the pluripotent stem cells of the inner cell mass of the embryonic blastocyst (see U.S. Pat. Nos. 5,843,780, 6,200,806, the contents of which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells.

In one embodiment, progenitor cells suitable for use in the claimed methods are Committed Ventricular Progenitor (CVP) cells as described in PCT Application No. WO 2010/042856, entitled “Tissue Engineered Mycocardium and Methods of Productions and Uses Thereof”, filed Oct. 9, 2009, the entire contents of which are incorporated herein by reference.

This invention is further illustrated by the following example which should not be construed as limiting. The contents of all references, patents and published patent applications cited throughout this application, as well as the Figures and Sequence Listing, are hereby incorporated by reference.

### Example. Focused Rotary Jet Spinning for the Biofabrication of Helically Aligned Heart Components and In Vitro Hydrodynamic Models of Hypertensive Remodeling

The goal of biofabrication is to engineer de novo tissue constructs which recapitulate the morphology and function of existing human organ systems. Achieving this would open up new options for patients facing end-stage organ failure and could improve the predicative capabilities of biomedical assays. Approaches using 3D extrusion printing have demonstrated important milestones, including fabricating microphysiological devices, microvasculature systems, and spontaneously beating hearts. However, engineering functional 3D biological structures with fine spatial features, complex geometries, and controlled alignments, while retaining high throughputs is challenging. This is the result of a fundamental tradeoff, where 3D extrusion printing speeds declines rapidly (power law, n=˜2.8) with respect to feature size, while the printing material required scales volumetrically with regard to organ size (FIG. 2A). Consider a full-sized human heart. Printing the collagen extracellular matrix components takes hours to days at current resolutions (E. Mirdamadi, et al., ACS Biomater. Sci. Eng. 6, 6453-6459 (2020)), but at the native feature size of a single micron, could potentially take hundreds of years following current trends in scaling. As a result, there is a need for approaches which can more rapidly produce single micron features, while retaining precise control over alignment and organization.

Micro/nanofiber spinning techniques offer a potential solution to this trade-off in reproducing fine spatial features in a high throughput manner. Methods including melt blowing, electrospinning, pull spinning, and centrifugal spinning, can form micro/nanoscale fibers, some of which offer orders-of-magnitude higher throughput than 3D extrusion printing. These techniques have already been used to engineer tissue constructs, such as heart valves (A. K. Capulli et al., Biomaterials. 133, 229-241 (2017)) and scale ventricle models (L. A. MacQueen et al., Nat. Biomed. Eng. (2018), doi:10.1038/s41551-018-0271-5.), suggesting that nanofiber based approaches may facilitate whole organ biofabrication. In practice, however, obtaining high quality fibers with precisely controlled alignments in complex 3D geometries has been difficult to achieve. In part, this is due to fiber formation and patterning being interrelated in many of these approaches, with fiber formation dictating material selection and subsequent deposition topologies. As described below, a new form of additive manufacturing, Focused Rotary Jet Spinning (FRJS), has been developed in which these two processes are decoupled, first producing fibers using rotary jet spinning and then concentrating these fibers into a focused air stream for deposition. This method allows for the rapid production of micro/nano sized fibers while providing control over fiber orientation and alignment. Using this approach, both helically and circumferentially aligned models of the left ventricle were manufactured, showing how these systems can be used to probe fundamental biomechanics and can scale to produce a full-sized model of the human heart.

The architecture of the human heart gives rise to its mechanical function, allowing it to efficiently pump blood to the rest of the body. In vivo, the left ventricle's myofibers are organized in a helical fashion, smoothly transitioning from a left-handed helix in the subepicardium to a right-handed helix in the subendocardium. These competing helical structures create a twist-based mechanism, allowing the heart to efficiently coordinate mechanical pumping. It has been argued that this helical organization is critical for achieving large ejection fractions (EF) with minimal myofiber shortening (Sengupta, P. P., et al. JACC Cardiovasc. Imaging 1, 366-376 (2008); Sallin, E. A. Biophys. J. 9, 954-964 (1969); Omar, A. M. S., et al. Circ. Cardiovasc. Imaging 8, 74-82 (2015); Stöhr, E. J., et al. Am. J. Physiol. Hear. Circ. Physiol. 311, H633-H644 (2016)). However, in some cardiomyopathies, such as hypertension (González, A. et al. Hypertension 72, 549-558 (2018); McCain, M. L., et l. Proc. Natl. Acad. Sci. 110, 9770-9775 (2013); Grossman, W., et al., J. Clin. Invest. 56, 56-64 (1975); Carruth, E. D. et al. J. Cardiovasc. Magn. Reson. 22, 21 (2020)), ischemia, or mitral valve regurgitation (Agger, P. et al. J. Cardiovasc. Magn. Reson. 19, 1-16 (2017)), this organization can be disrupted by maladaptive tissue remodeling (McCain, M. L., et al. Proc. Natl. Acad. Sci. 110, 9770-9775 (2013); Hung, C. L. et al. J. Am. Coll. Cardiol. 56, 1812-1822 (2010)), potentially leading to more circumferentially aligned myofibirals (Grossman, W., et al. J. Clin. Invest. 56, 56-64 (1975); Carruth, E. D. et al. J. Cardiovasc. Magn. Reson. 22, 21 (2020); Agger, P. et al. J. Cardiovasc. Magn. Reson. 19, 1-16 (2017); Hung, C. L. et al. J. Am. Coll. Cardiol. 56, 1812-1822 (2010)). In addition to these structural differences, most cardiomyopathies are also accompanied by changes in protein expression, and metabolism (Zhang, J. Clin. Exp. Pharmacol. Physiol. 29, 351-359 (2002), making it difficult to decouple how mechanical and biochemical ques individually contribute to a given cardiomyopathy. As a result, there is still a need for improved in vitro models to probe how the biomechanics of hypertensive remodeling contribute to cardiac failure.

To date, studies focusing on the mechanics of ventricular remodeling have primarily been conducted in silico or in vivo. As early as the 1960's, analytical models emerged describing the heart's twisting motion (Sallin, E. A. Biophys. J. 9, 954-964 (1969). More recently, expanded analytical (Voorhees, A. P. & Han, H. C. Theor. Biol. Med. Model. 11, 1-19 (2014); Triposkiadis, F. et al., Eur. J. Heart Fail. 20, 436-444 (2018)) and computational models (Baillargeon, B., et al. Eur. J. Mech. A/Solids 48, 38-47 (2014); Doenst, T. et al. Ann. Thorac. Surg. 87, 1187-1195 (2009)) have been employed, incorporating features such as myofibril alignment, tissue stress-strain relationships, and fluid displacement. Collectively, this has suggested that helical ventricles should display increased major axis shortening and more uniform strain distributions, with greater ejection fractions (Hung, C. L. et al. J. Am. Coll. Cardiol. 56, 1812-1822 (2010)). Confirming these predictions experimentally, however, has proven difficult. In the case of in vivo systems, pulmonary valve regurgitation (Agger, P. et al. J. Cardiovasc. Magn. Reson. 19, 1-16 (2017)), or transverse aortic constriction (Carruth, E. D. et al. J. Cardiovasc. Magn. Reson. 22, 21 (2020)) can be used to induce more circumferential alignments. Additionally, the spontaneously hypertensive rat models show changes in ventricle architecture (Gonzilez, A. et al. Hypertension 72, 549-558 (2018); Tran, N., et al. J. Med. Imaging 3, 046001-046001 (2016), and a reductions in ejection fraction (Pfeffer, J. M., et al. Proc. Natl. Acad. Sci. U.S.A. 79, 3310-3314 (1982). However, precisely controlling ventricle alignment can be challenging. Additionally, animal models present similar complications to human patients, where myopathies emerge as a result of both biochemical and mechanical cues. Improved in vitro models have the potential to help disentangle these competing factors. In this regard, researchers have recently shown the ability to produce stand-alone ventricle scaffolds (Lee, A. et al. Science (80-). 365, 482-487 (2019); Noor, N. et al. Adv. Sci. 6, 1900344 (2019); Yoo, S.-J. et al. 3D Print. Med. 2, 3 (2016)) using methods such as 3D printing. These engineered tissue constructs are promising as they allow for the recapitulation of broad anatomically features, can contract in vitro, and show calcium propagation. However, to measure the impact of myofibril architecture, more precise templating is needed at the micron length scale to control tissue alignment.

Spun nanofiber ventricles offer a potential solution to these problems (MacQueen, L. A. et al. Nat. Biomed. Eng. (2018). doi:10.1038/s41551-018-0271-5), as nanofibers can provide mechanical support while acting as topological guides to tissue reconstruction. Additionally, their relative orientation can be selected during scaffold manufacture, making them amenable to constructing controlled ventricle orientations. Here it is proposed to use 3D ventricle scaffolds as tissue engineered in vitro models to probe how myofibril alignment can lead to altered cardiac output. Based on computational and experimental models, it was determined that a helical angle of ˜30° in ventricle models can produce maximal thrust, resulting in greater ejection fractions. To test this hypothesis, both helically (30°) and circumferentially (0°) aligned 3D nanofiber ventricle scaffolds seeded with cardiomyocytes were produced, seeking to recapitulate a wild-type and hypertensive heart. Helically aligned ventricles showed more uniform strains, along with increased axial shortening, cardiac output, and ejection fractions. Overall, this work provides new experimental techniques for probing the mechanisms of ventricle twist. Additionally, this works represents the first exploration of fluid-dynamics in an in vitro ventricle model, helping bridge the gap between laboratory studies and clinically relevant functional readouts.

### A. Fiber Manufacture Using Focused Rotary Jet Spinning

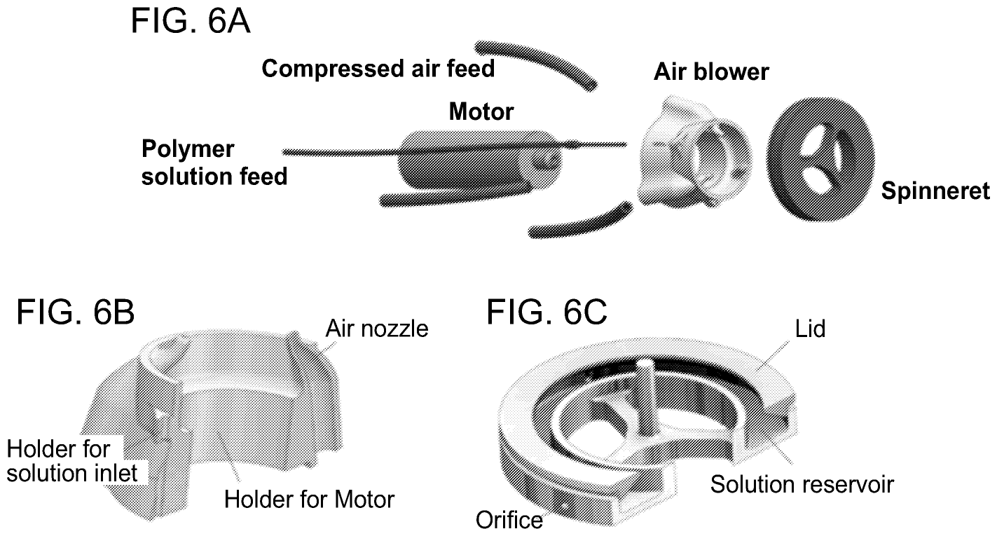

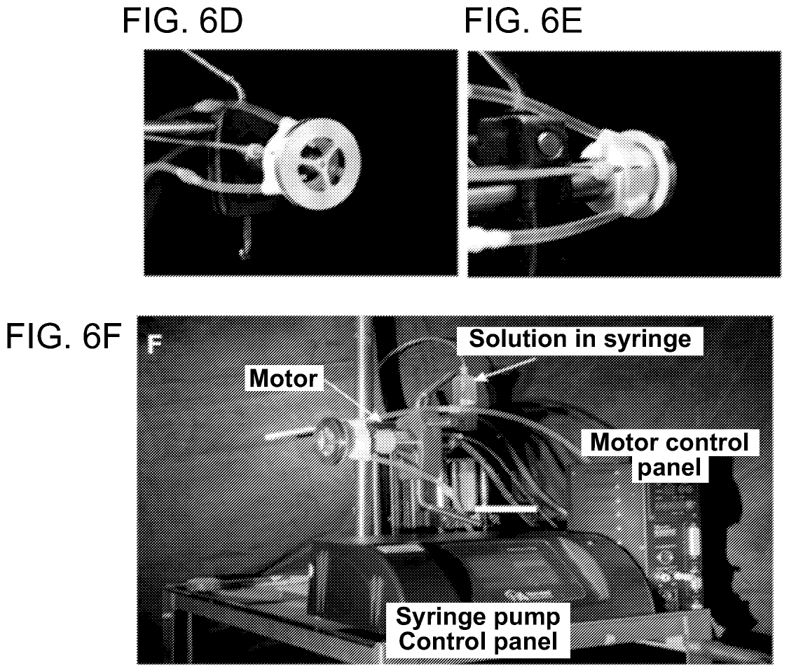

In FRJS, decoupling the fiber formation and patterning processes is realized by creating a focused stream of pre-formed fibers (FIG. 2B, FIG. 6). Rotary jet spinning produces fibers by centrifugal force, generating a cloud of fibers surrounding the spinneret (FIG. 2C). This allows for an independent formation period, where fibers undergo jet elongation, resulting in free-floating single micron fibers. Next, in a phenomenon known as entrainment, fibers are pulled into a jet stream blown from the center of the spinneret. The entrainment flow is orders of magnitude slower than the air inside the jet, allowing it to minimally perturb fiber formation. Entrainment causes fibers to converge and accelerate towards the jet, which confines and aligns them into a continuous focused stream. To verify this focusing effect, polycaprolactone (PCL) fibers were collected at regular intervals within the airstream to measure deposition profiles. This showed that, at the narrowest point, 95% of fiber deposition occurred within a 5.0±0.3 cm spot size (2a of Gaussian distribution) (FIG. 2D).

Next, how fiber and subsequent tissue alignment can be controlled using FRJS was examined. It was hypothesized that the alignment of fibers in the air stream could enable controlled fiber deposition, where tangential collection (θ=0°) should minimally perturb airflows, while head-on deposition (—0=90°) should lead to divergent patterning. To confirm this, the relative collector orientation was modulated during fiber deposition (FIG. 3A). The resulting fibers displayed angle dependent anisotropy, with tangential collection leading to highly anisotropic fibers (θ=0°, Orientation Order Parameter, OOP=0.60±0.15), perpendicular collection displaying random fiber orientations (θ=90°, OOP=0.18±0.03), and intermediate incident angles leading to partially aligned configurations (θ=60°, OOP=0.50±0.08). As an additive manufacturing technique, this suggested that more complex patterning could be achieved by moving the target relative to the stream. For example, it was demonstrated that collecting on an incrementally rotating disk generates a multilayered fiber sheet with controllable fiber orientations as confirmed by x-ray microtomography (PCT) (FIG. 3B). Additionally, collecting on an inclined rotating cylinder generated helical alignments, which have been difficult to achieve at the micron scale using current fiber patterning approaches (FIG. 3C-F). To ensure that FRJS could be used to direct cell infiltration and orientation, gelatin nanofiber scaffolds were also seeded with cardiomyocytes, resulting in highly anisotropic tissues organized in the direction of fiber alignment (FIG. 3G). This diversity in manufacturing and ability to direct tissue alignment indicated that FRJS could be used in a hierarchical manner to assist in biofabrication. Models of the heart were generated, using the left ventricle as an exemplar of how tissue alignment impacts organ function.

### B. Helically Aligned Model of the Left Ventricle

In healthy patients, the heart is composed of multiple layers of cardiomyocytes, with alignment of cardiomyocytes in the ventricle rotating transmurally from helical angles of +60° to −60° relative to circumferentially aligned mid-layer (Ferreira. P. F. et al. J. Cardiovasc. Magn. Reson. 16, 1-16 (2014)). As also noted above, in some cardiomyopathies, such as hypertension, ischemia, or mitral valve regurgitation, this organization can be disrupted by maladaptive tissue remodeling, potentially leading to more circumferentially aligned myofibrils. For more than fifty years, it has been argued that this helical structure is critical for achieving large ejection fractions (EF) (P. P. Sengupta, A. J. Tajik, K. Chandrasekaran, B. K. Khandheria, JACC Cardiovasc. Imaging. 1, 366-376 (2008); E. A. Sallin, Biophys. J. 9, 954-964 (1969); A. Grosberg, M. Gharib, A. Kheradvar, Bull. Math. Biol. 71, 1580-1598 (2009)). However, testing this fundamental assumption has been limited by the ability to recreate these native architectures.

Given FRJS's ability to facilely control nanofiber alignment, it was reasoned this approach could be used to create simple helically aligned ventricle scaffolds. To examine how these aligned components can give rise to organ function, a simplified in vitro model incorporating single helical angle was made, and then used to examine how differing tissue alignments can lead to changes in pumping efficiency. These controlled systems, specifically three-dimensional engineered tissue ventricle models, provide a platform to examine how ventricle alignment contributes to cardiac performance, and serve as an important steppingstone towards achieving full-organ biofabrication.

To simplify this system and decompose these structures into a single helical angle which can be achieved in vitro. Fiber orientations were selected based on the average myofibril dispersion angle (34.8°) of a healthy heart, which measures the variation in local fiber orientation with respect to the mean fiber orientation (Tran N, Giannakidis A, Gullberg G T, Seo Y. Quantitative analysis of hypertrophic myocardium using diffusion tensor magnetic resonance imaging. J Med Imaging (Bellingham). 2016 October; 3(4):046001. doi: 10.1117/1.JMI.3.4.046001. Epub 2016 Nov. 3. PMID: 27872872; PMCID: PMC5093228). Given these conditions it was hypothesized that helically aligned ventricles would show increased cardiac output and EF. To test this hypothesis and to demonstrate that alignment can lead to functional differences in tissue performance, both circumferentially aligned (CA, α=0°) and helically aligned (HA, α=30°) three-dimensional gelatin fiber tissue scaffolds, representing hypertensive and healthy patients, respectively, were produced. As noted above, helically aligned (HA) fiber orientations were selected, in part, based on the average myofibril dispersion in wild-type (healthy) rodent hearts (34.8°).

As previously reported, NRVMs seeded onto linearly patterned fibronectin surfaces will ‘read’ the underlying mechanical cues provided by the ECM, self-organizing into laminar anisotropic tissues, with aligned sarcomeric α-actinin. Previously, this approach has been used to control features such as tissue architecture and cell alignment (Nawroth, J. C. et al. Nat. Biotechnol. 30, 792-7 (2012); Park, S.-J. et al. Science (80-.). 353, 158-162 (2016)), providing insights into myocardial dysfunction (McCain, M. L., et al. Proc. Natl. Acad. Sci. 110, 9770-9775 (2013); Wang, G. et al. Nat. Med. 20, 616-623 (2014)). Similarly, tissue scaffolds including oriented micron and nanometer-diameter polymeric fibers were used to guide tissue architecture in the model ventricle.

Focused rotary jet spinning (FRJS) was used to produce a scale model of the left ventricle, with helical and circumferential fiber orientations (FIGS. 4A-B, 15a). As explained above, FRJS works by ejecting a thin liquid jet, in this case dissolved gelatin, into a concentrated airstream. As the liquid is pulled through the air, solvent rapidly evaporates off causing the gelatin to coalesce into a solid fiber. The spun nanofibers are then collected onto a rotating mandrel in the shape of a ventricle (FIGS. 15a-15B). As explained above, by controlling the mandrel's tilt angle and orientation, fibers can be organized either helically or circumferentially, guiding subsequent cell infiltration and tissue alignment. Additionally, it has been shown that nanofiber scaffolds seeded with cardiomyocytes can serve as a scale model for cardiac function (MacQueen, L. A. et al. Nat. Biomed. Eng. (2018). doi:10.1038/s41551-018-0271-5). Combining these insights, it was then sought to examine how hypertensive remodeling can impact clinically relevant functional readouts.

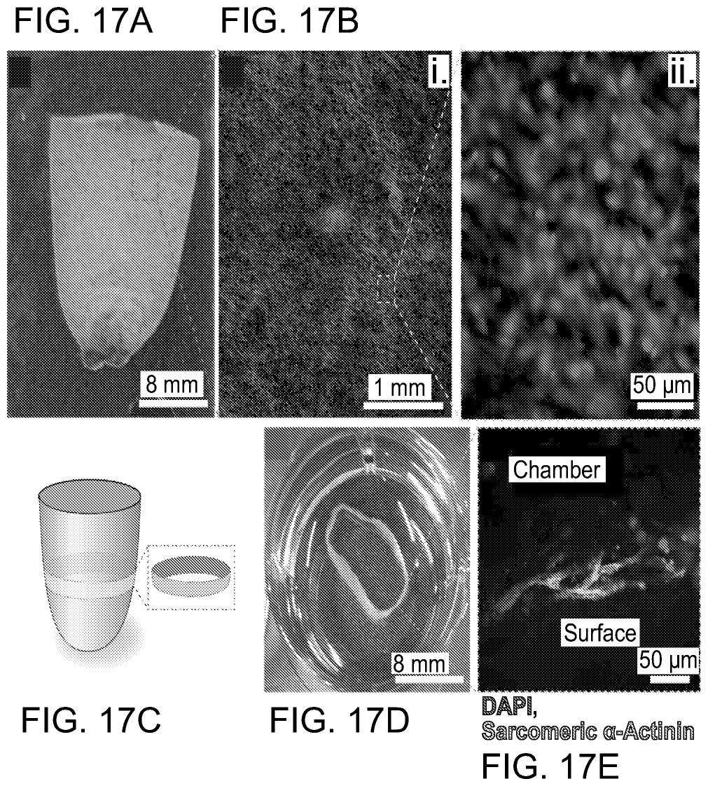

Mimicking the hypertensive and healthy states, both CA and HA ventricle-shaped scaffolds were seeded with Neonatal Rat Ventricular Myocytes (NRVMs), forming aligned and continuous tissue segments (FIGS. 4C, 15C, 17). Synchronous, coordinated ventricle contractions emerged spontaneously within three days and persisted for the duration of tissue culture, with experiments being performed five to six days after seeding.

Previously it has been hypothesized that HA ventricles would display increased apical shortening (E. A. Sallin, Biophys. J. 9, 954-964 (1969); N. B. Ingels, Technol. Heal. Care. 5, 45-52 (1997)), and reduced basal strain (N. B. Ingels, Technol. Heal. Care. 5, 45-52 (1997)), with helical angles leading to a more distributed strains. Additionally, hypertensive remodeling is believed to lead to increased ventricular strain, with helical angles leading to a more distributed load across the ventricle surface (Ingels, N. B. Technol. Heal. Care 5, 45-52 (1997)). To investigate these claims using the model system described herein, how scaffolds of the engineered tissue ventricles deformed during stimulated contraction was first examined.

### C. Ventricle Strain and Deformation

Fiber deformation was visualized by submerging cardiomyocyte seeded ventricle scaffolds into a solution containing fluorescent beads for fifteen minutes, resulting in the fluorescent beads non-specifically adhering to the surface of fibers in the scaffolds (FIG. 15D). Samples were then transferred to a fresh Tyrodes solution, field stimulated, and imaged using a fluorescent microscope (FIG. 15E). Using digital image cross-correlation to measure deformations in the fiber structure, strain mapping was performed over the ventricle surface (FIGS. 4E, 15F). CA ventricles displayed greater strains near the base of the ventricle, while HA ventricles showed more uniform deformations, with increased strain at the ventricle's apex (FIGS. 4D, 4E, 15F).

To probe this further, changes in the ventricle's boundary resulting from strain were examined (FIGS. 7a-7b). Greater basal deformation was observed in the CA scaffold, with increased apical deformation in the HA scaffolds. Quantifying these differences using an elliptical fit normalized by the total area change, showed significant differences in ventricle deformation for the different alignments (FIGS. 7b-7d), observing significant differences in deformation between each alignment. CA ventricles displayed greater minor-axis shortening, with only minimal changes occurring in the major axis. Conversely, HA ventricles displayed significant major axial shortening, with reduced basal deformations. Where CA samples showed greater basal and minimal longitudinal constriction, HA ventricles displayed significant apical shortening with reduced basal deformation, resulting in more uniform strains (FIGS. 7a-7d).

Overall, these results were consistent with previous analytical models, indicating that alterations in the underlying tissue alignment can potentiate changes at the organ scale. These results also corroborate reports of increased circumferential strain in hypertensive patients (Liu, H. et al. Sci. Rep. 10, 1-9 (2020)), suggesting that a reduction in helical architectures can lead to increased localized strains in the pathological case.

### D. Alignment Dictates Calcium Wave Propagation

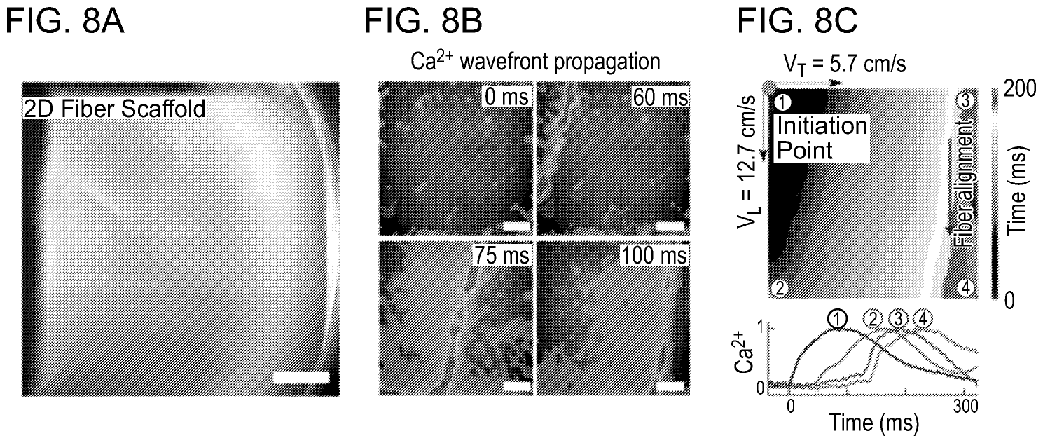

Next, the impact of fiber orientation on calcium wave propagation was examined. In healthy tissues, action potentials propagate faster in the direction of cell alignment, with myocytes showing reduced conduction velocities (CV) in the transverse direction. Given this biophysical insight, it was reasoned that electrical signal propagation may vary depending on the ventricle's fiber alignment. First, to show that anisotropic signal conduction in tissue scaffolds, 2D gelatin fiber constructs were point stimulated and the resulting calcium signaling was recorded using an optical mapping system. This confirmed increased CVs in the direction of fiber alignment, with longitudinal to transverse CV ratios of ˜1.5-2.0 (FIG. 8A-8C). Turning to 3D models, ventricles with extended basal regions-allowing for increased travel distances—were apically stimulated. Isochrones of the calcium propagation reveal that CA scaffolds displayed only modest CVs (8.3 cm/s) along the ventricle's long axis, while HA ventricles showed greatly increased CVs (19.1 cm/s), with signals taking less than half the time to transverse the ventricle's surface (FIG. 4F). This confirmed that fiber orientation plays an important role in regulating calcium signaling in the model system described, further demonstrating the importance of myofibril alignment in healthy cardiac function.

### E. Ventricle Twist

Helical myofibril architectures are also believed to give rise to rotational displacements, or ventricular twists (P. P. Sengupta, A. J. Tajik, K. Chandrasekaran, B. K. Khandheria, JACC Cardiovasc. Imaging. 1, 366-376 (2008); A. M. S. Omar, S. Vallabhajosyula, P. P. Sengupta, Circ. Cardiovasc. Imaging. 8, 74-82 (2015)). In a healthy heart, kinetic energy is stored during contraction in the sarcomeric protein titin and is subsequently released during early myofiber relaxation (A. González et al., Hypertension. 72, 549-558 (2018); A. M. S. Omar, S. Vallabhajosyula, P. P. Sengupta, Circ. Cardiovasc. Imaging. 8, 74-82 (2015); E. J. Stöhr, R. E. Shave, A. L. Baggish, R. B. Weiner, Am. J. Physiol. —Hear. Circ. Physiol. 311, H633-H644 (2016)). This aides in the rapid uncoiling of fibers and helps produce diastolic suction. As a result, ventricle twist is thought to provide a key mechanistic link between systolic and diastolic function. To study if twist was preserved in the model system described herein, rotational displacement at the apex of suspended ventricle scaffolds was measured (FIGS. 9A-9E). Here both HA and CA scaffolds were sutured at the base to a fixed support, and were monitored from below during field stimulation. Rotation was measured based on rotation of an edge or perimeter of a cross-section of the ventricles. The perimeter or edge of the cross-section was fitted using an elliptical fit, and rotation was measured as a change in an orientation of the major axis of the ellipse. The CA scaffolds showed minimal torsion (1.35±1.1°) (FIGS. 9D-9E). Conversely, HA scaffolds showed an approximately four times greater rotational displacement (5.4±3.6°) (FIGS. 9D-9E). Combined with the changes in deformation (e.g., strain and axial shortening), and calcium propagation these rotational displacements, indicated substantive structural differences across scaffold orientations, indicating the potential for functional differences in ejection velocities and cardiac output for different scaffold orientations, such as potentially increased ejection velocities and cardiac output in the helical case.

### F. Cardiac Output and Ejection Fraction

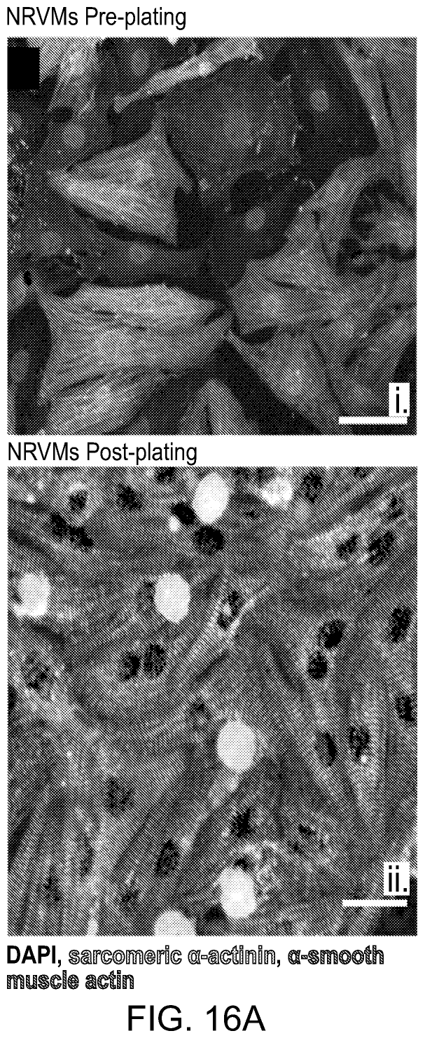

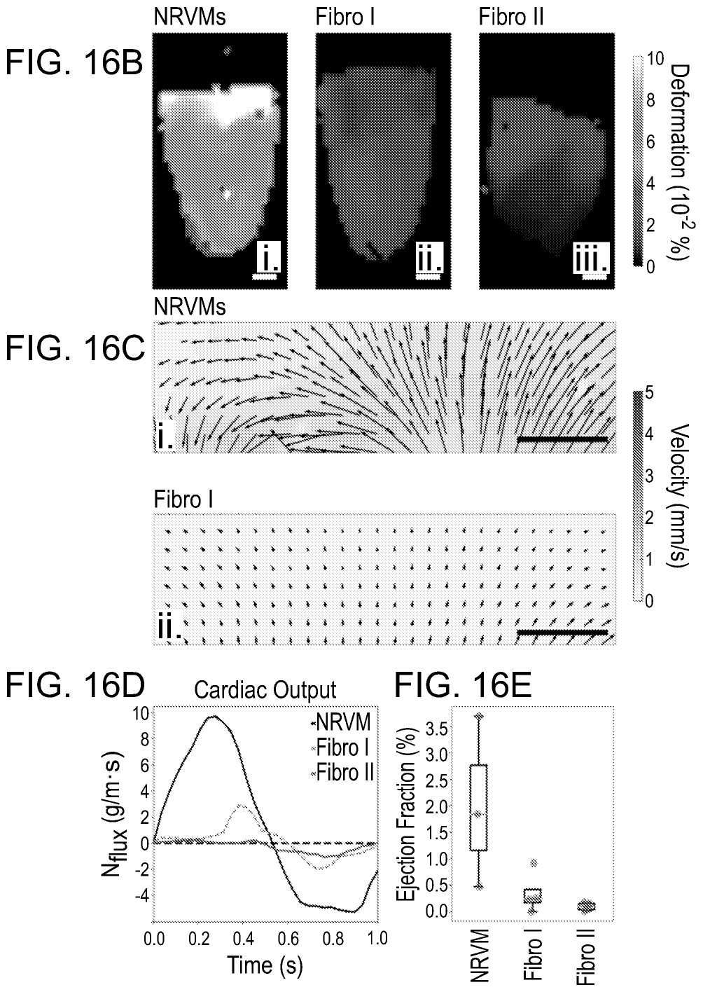

Both ejection fraction (EF), which is the fraction of blood expelled by the ventricle during systole, and cardiac output (CO), which is the volume of blood transferred, are used as common metrics of cardiac health. To examine how structural changes in alignment and deformation could lead to functional differences, such as altered cardiac performance, Particle Imaging Velocimetry (PIV) was used to quantify changes in ejection velocities and cardiac output (A. Liberzon, et al. OpenPIV/python (2019), doi:10.5281/zenodo.3566451). Ventricle constructs were suspended in a bath containing neutrally buoyant fluorescent beads (FIG. 10A), and bead displacement was tracked (FIGS. 10B, 10C). This allowed for the construction of two-dimensional (2D) velocity fields surrounding the basal opening, as shown for a HA ventricle model. A trace of the fluorescent particles near the base of the ventricles, revealed larger field lines in the helical case (α=30°), with only minimal movement for CA scaffolds. Quantifying this further, two-dimensional (2D) velocity fields surrounding the basal opening of the ventricle constructs were determined using tracked bead displacements in the selected region of interest (ROI) surrounding the basal opening using PIV, thereby measuring instantaneous changes in fluid velocity during stimulation (FIG. 4G). This was performed for ventricles both with varying fiber alignments, and fibroblast compositions (FIG. 16). In the model system described herein, both, fluid being expelled during systolic contraction, and refill occurring due to diastolic suction could be observed. This indicated the ability of the model ventricle system to act as a functional fluidic pump.

The instantaneous mass flux resulting from ventricle contraction and diastolic refill were also evaluated (FIGS. 4H, 10D-10E), and cyclic outputs, with fluid being expelled during systolic contraction, and refill occurring during diastole were determined (FIG. 4H, 10F).

Summing or integrating the fluid displacement over systolic period yielded the total cardiac output, with values of 1.3±0.9 g/m and 2.6±1.3 g/m for CA and HA ventricle scaffolds, respectively (FIG. 4I). This represented a significant (p<0.05) increase in cardiac output based purely on ventricular tissue alignment.

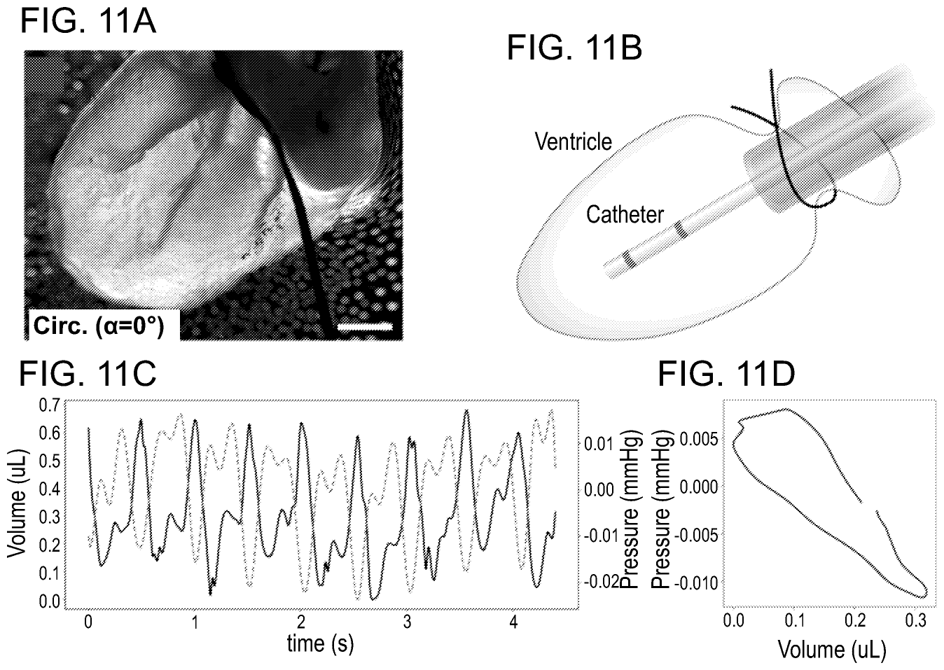

To measure ejection fractions (EF), pressure-volume (PV) changes in the ventricle scaffolds using catheterization were monitored, observing the formation of complete oblate PV loops (FIG. 11A-11D). However, submerged gelatin nanofibers are a poor dielectric, making it difficult to obtain consistent results in these synthetic scaffolds using conductance catheterization.

By integrating the cardiac output over the ventricular opening and then normalizing by the original mandrel volume and fluid density, cardiac EFs were estimated (FIG. 4I). Here an increase in the EF for HA ventricle scaffolds was observed, with an average EF of 1.6±1.1% and 3.3±1.7% in the CA and HA scaffolds, respectively, with a maximum EF of 5.9% observed in the helical scaffold. Although these values are significantly lower than in vivo EFs (˜60-80%), this is expected given that the model system described herein is composed of a monolayer of cardiomyocytes, and these values are consistent with previously reported in vitro systems (L. A. MacQueen et al., Nat. Biomed. Eng. (2018), doi:10.1038/s41551-018-0271-5; M. E. Kupfer et al., Circ. Res., 207-224 (2020)11)). With a ˜100% increase in the helical case, these results indicate that tissue alignment plays a critical role in modulating ventricular ejection fractions. Overall, these results demonstrate that ventricle geometries play an important role in determining cardiac ejection fractions.

### G. Dual Chamber Ventricles and Full-Scale Heart Models

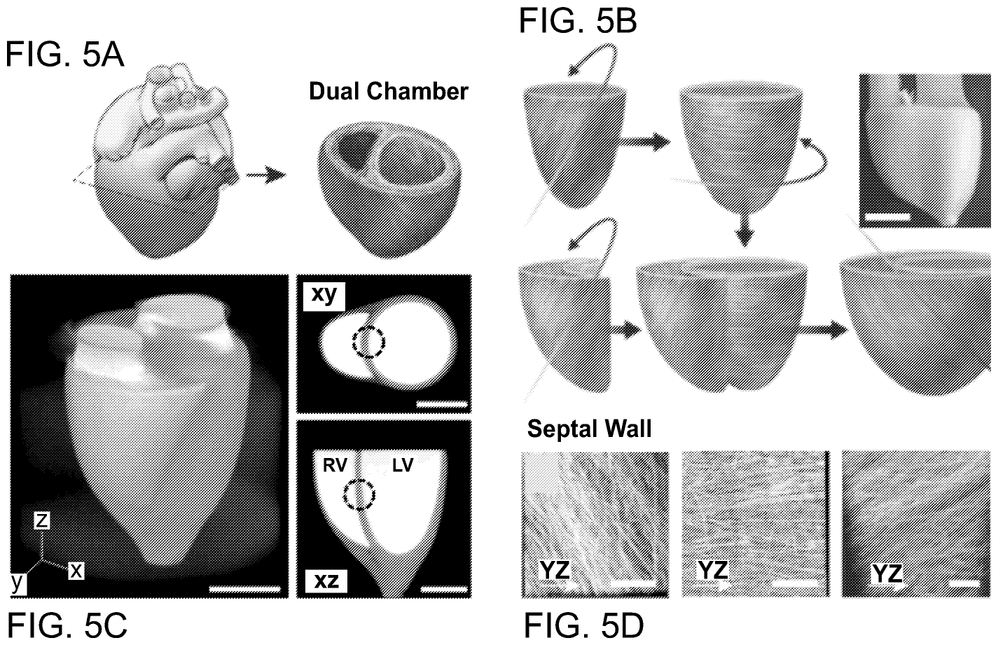

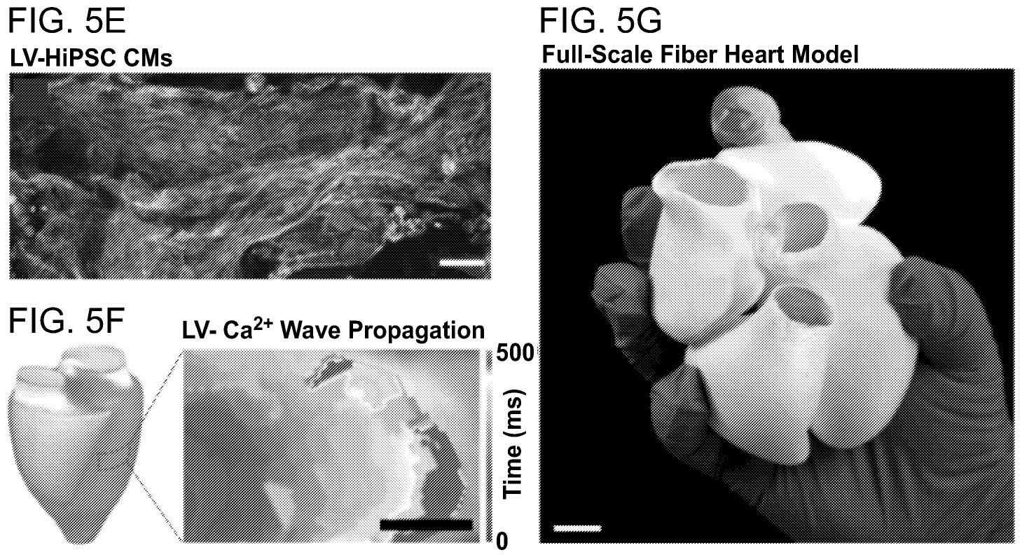

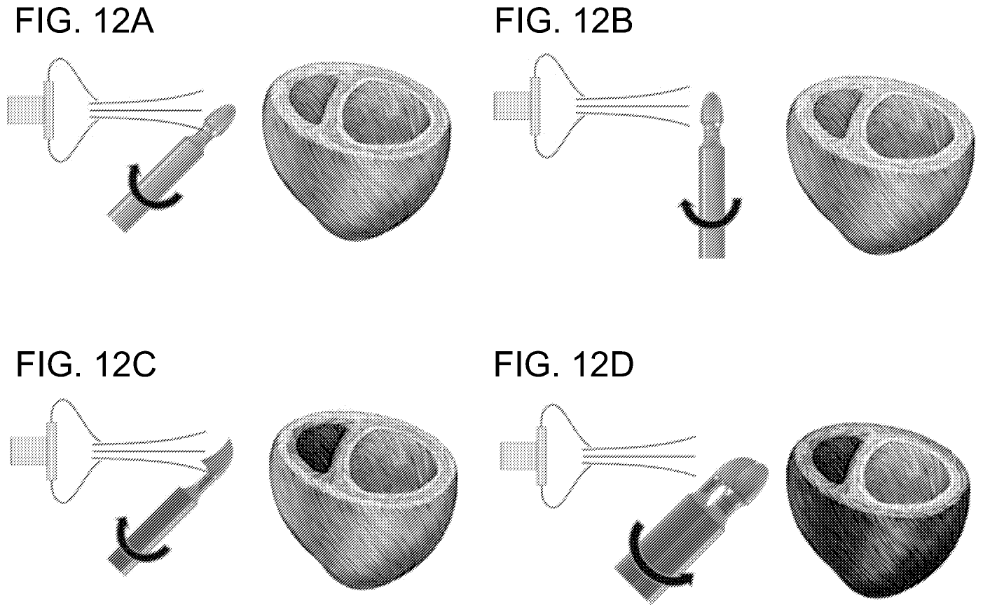

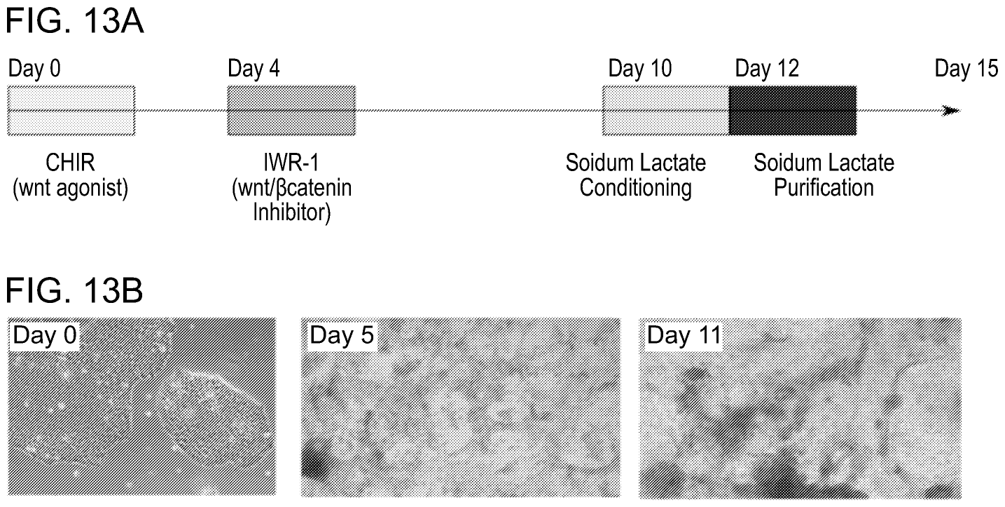

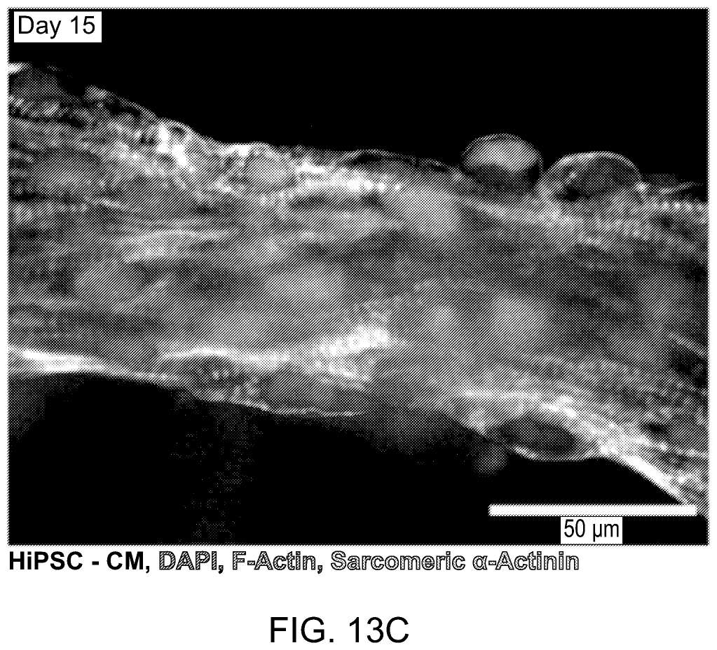

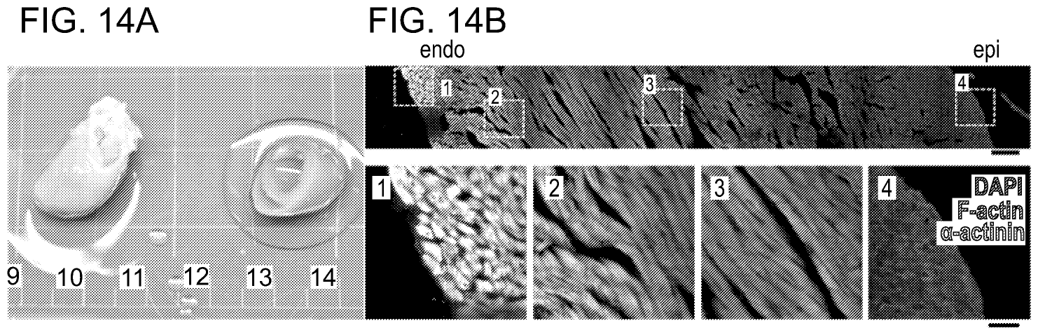

Moving towards higher fidelity representations of the human heart, FRJS was then used to create models with multiple helical angles, including a Dual-Chambered Ventricle (DCV) and a full-scale human heart model (FIG. 5A-5G). DCVs were manufactured using a multistage process, creating inner and outer helical layers, with an intermediate circumferential sheet reminiscent of native myocardium (FIGS. 12A-12D, FIG. 5B-D). These competing helical structures were confirmed using μCT, indicating three distinct transmural layers along the septal wall (FIG. 5C-D). Showing that DCVs could support cardiac tissues, scaffolds were then seeded with human stem-cell derived cardiomyocytes (HIPSC-CMs), which were differentiated in vitro, forming contractile tissues exhibiting visible spontaneous contractions after seven days, and staining positive for sarcomeric α-actinin (FIGS. 13A-13C).

Examining an excised segment of the left-ventricle it was observed that cells adhered to the fibers, forming aligned cardiac tissues (FIG. 5E). In the absence of vascularization, tissue infiltration was primarily restricted to the scaffold surface. Additionally, calcium imaging revealed sustained wave propagation across the ventricle surface, occurring primarily in the direction of fiber alignment, indicating the formation of a continuous cardiac syncytium (FIG. 5F). Overall, this demonstrated that fiber constructs can support human stem cell derived tissues with complex geometric structures and controlled alignments

To demonstrate the scalability of FRJS biofabrication, a full-sized human heart model was constructed. This was assembled by individually patterning micro/nanoscale fibers onto dissolvable collectors in the shape of each of the heart's four chambers. Individual chambers were then connected by an exterior fiber shell, before dissolving the interior supports, resulting in a standalone scaffold construct (FIG. 5G). While full-sized anatomical models have previously been produced using thermoplastics and hydrogels (E. Mirdamadi, J. W. Tashman, D. J. Shiwarski, R. N. Palchesko, A. W. Feinberg, ACS Biomater. Sci. Eng. 6, 6453-6459 (2020)), these systems typically lack the micron-scale architecture needed to direct cell alignment. Here, using a nano/microfiber based approach, these local structures can be preserved across entire tissue volumes, allowing for the hierarchical assembly of tissue constructs. These 3D structures demonstrated that FRJS based scaffolds can support human derived tissues with multiple alignments, while allowing for the rapid assembly of full-sized models systems. These key features mark fiber-based manufacturing as an approach for achieving whole organ biofabrication, which can be used as an alternative to or in conjunction with 3D printing.

### SUMMARY

As described in this example, a novel approach for biofabrication has been described using FRJS to overcome trade-offs in spatial resolution and manufacturing throughput. This allowed for the rapid assembly of 3D ventricle models capable of recapitulating emergent phenomena in vitro, including ventricular twist, strain displacement, and ejection fractions. By providing clinically relevant metrics these model systems provide for improving the predicative capabilities of biomedical assays. To demonstrate this, it has been shown how cardiac myofibril alignment regulates ventricular performance, confirming theoretical predictions made over fifty years prior (E. A. Sallin, Biophys. J. 9, 954-964 (1969)). These results demonstrate that a reduction in helical architecture can lead to altered cardiac performance in some pathological case, reminiscent of increased circumferential strain (H. Liu et al., Sci. Rep. 10, 1-9 (2020)) and reduced ejection fractions in hypertensive patients, where maladaptive tissue-remodeling can lead to more circumferential alignments (A. Gonzilez et al., Hypertension. 72, 549-558 (2018); M. L. McCain, S. P. Sheehy, A. Grosberg, J. A. Goss, K. K. Parker, Proc. Natl. Acad. Sci. 110, 9770-9775 (2013); W. Grossman, D. Jones, L. P. McLaurin, J. Clin. Invest. 56, 56-64 (1975); E. D. Carruth et al., J. Cardiovasc. Magn. Reson. 22, 21 (2020)).

An in-vitro model which can serve as microphysiological systems for hypertensive remodeling was presented using a 3D scaffold system to demonstrate how these changes impact cardiac output and ejection fraction. In using these ventricle models, it should be noted that the angles selected represent more extreme differences than those presented in clinical remodeling, which typically present as only a few degrees of difference (Tran, N., et al. J. Med. Imaging 3, 046001-046001 (2016)); however in vivo, these small changes occur over several layers of cardiomyocytes, amplifying their effects as compared to this single layered model. Additionally, by using this simplified approach, the pathophysiological consequences of ventricular remodeling were illustrated.

These systems can also be used as drug screening platforms. While this example has focused primarily on geometric and mechanical contributions to ventricle performance, these models can also be used for testing therapeutic agents, more closely linking in vitro measurements with clinically relevant readouts. For example, the three-dimensional tissue ventricle models can be used as a tool for studying changes in contractile force in response to pharmaceuticals, toxins, diet supplements, pollutants, and other endogenous factors (e.g. hormones, metabolic products, etc.), providing insight into several model disease systems. Similarly, as the 3D ventricle model recapitulates unique behaviors such as ventricular twist, and strain displacement in vitro, this platform serves as important resource for therapeutics targeting these readouts. One example for identifying potential therapeutic interventions includes akinetic or dyskinetic myocardium resulting from myocardial infarction, which have been linked with increased wall stress (Klein, P. et al. Eur. J. Heart Fail. 21, 1638-1650 (2019)) and reduced ventricle twist (Onohara, D. et al. J. Thorac. Cardiovasc. Surg. 30313, (2020)).

In addition to providing a platform for improved clinical readouts, these models also support emerging clinical approaches using conical ventricle reshaping to address heart failure (Klein, P. et al. Eur. J. Heart Fail. 21, 1638-1650 (2019)). Although the present study has focused on the long-term effects of hypertensive ventricular remodeling, it has been shown the ventricle alignment plays a crucial role in governing cardiac output and ejection fraction. This demonstrates that recovering this alignment in the wake of cardiac failure is critical in restoring proper ventricle function. Recently, ventricle reshaping has been viewed as controversial due to a landmark surgical study showing that despite achieving improvements in left ventricle volumes, there was no significant increase in survival, hospitalization, or functional outputs with ventricular reconstruction (Jones, R. H. et al. N. Engl. J. Med. 360, 1705-1717 (2009)). However, subsequent studies have argued that this is a result of focusing on solely on ventricle volume, rather than the underlying conical ventricle shape (Onohara, D. e, al. J. Thorac. Cardiovasc. Surg, 30313, (2020); Calafiore, A. M. et al. Eur. J. Cardio-thoracic Surg. 50, 693-701 (2016). The results herein lend credence to this idea, suggesting that in addition to the conical shape, the final myofibril architecture may also play a critical role. As a result, this may explain why studies in this field present conflicting results, suggesting that special consideration should be given to the final tissue orientation and alignment post-reconstructive surgery. Taken together, this demonstrates that the presented micro physiological systems offer potential insights into cardiac physiology and provide a suitable test system for performing in vitro studies that increasingly translate to improved clinical outcomes.

### Materials and Methods

The following Materials and Methods were used in the Example.

### Focused Rotary Jet Spinning

A high-speed motor spindle (NR-4040, part No. NSK 9213) was purchased from Nakanishi, including all related parts: collect chuck (CHK-6.35AA, part No. NSK 91601), collect nut (part No. NSK 2158), control unit (E3000, NE 211, part No. NSK 9775), motor cord (EMCD-3000J-4M, part No. NSK 1768), air-line kit (AL-A1205, part No. NSK 4505), and controller extension (E3000-PEX4, part No. NSK 8407)

The air blower used to form the fiber stream was 3D printed from an Objet30 3D printer (Stratasys, Eden Prairie, Minnesota, USA) using VeroWhitePlus® (RGD835) photopolymer resin, Fig.S2B (see FIGS. 6A and 6B). The air blower is press-fitted onto a motor spindle and is connected to the building's compressed air source through an air pressure regulator (part No. NSK 4505, Nakanishi)

The spinneret was machined from aluminum 7075, and is 6.4 cm in diameter. The spinneret design contains three orifices, each 400 μm in diameter (see FIGS. 6A and 6C). The spinneret lid was machined from polypropylene for its solvent resistance, and was press-fitted onto the bottom to ensure proper sealing.

The polymer solution was fed into the spinneret through a small needle, which was fitted in place into a small hole in the air blower. Using an automated syringe pump, purchased from Harvard Apparatus (part No. 703007), polymer solution was passed between a syringe (60 mL, Luer-Lok™ Tip) and the needle (BD company) via flexible tubing (see FIGS. 6A and 6D-6F). The Polyfluoroalkoxy alkane tubing was purchased from Saint-Gobain (Part No. TSPF35-0125-031-50). The luer lock couplings (part No. 51525K326 and part No. 51525K24) was obtained from McMaster-Carr company.

Universal stand (part No. 570-12000-00) and clamps (part No. 36300550) holding the motor were purchased from Heidolph Instruments.

For fiber collection, the collection mold was fixed into a high-speed motor spindle (NR-4040, part No. NSK 9213) with a Nakanishi E4000 controller.

### Solution Preparation and Fiber Spinning

Polycaprolactone (PCL, average Mn=80,000, Sigma-Aldrich 440744) was used as the default polymer in this study, unless otherwise specified. Hexfluoroispropanol (HFIP, Oakwood Chemical 003409) was used as the primary solvent, unless otherwise specified. The polymer was dissolved in HFIP solvent at room temperature on a magnet stir plate (PC-4200 Corning) overnight. The concentration of 6 g/100 ml PCL solution, 6 g/100 ml Nylon 6 pellets (Sigma-Aldrich 181110) and 5 g/100 mL Gelatin type A (Sigma-Aldrich G2500) were prepared and used in corresponding experiments.

### Polyurethane Fiber Preparation Method

For fiber spinning, rotation speed of the spinneret was set to 10,000 RPM and the pressure input to the air blower was 0.2 MPa unless otherwise specified. The number of orifices in spinneret, solution flow rate, total solution volume and collection distance were described in each experiment. All collection targets were treated with Teflon spray (DuPont, Teflon Non-Stick Dry-Film Lubricant) before deposition. Two of the three orifice on the spinneret were used for spinning unless otherwise specified. A KAPTON tape (¼″ Wide, 15′ Long, 0.0025″ Overall Thickness, part No. 7648A731, McMaster-Carr) was used to block the orifice. Polymer solution was fed into the spinneret at 0.4 ml/min unless otherwise specified.

Fiber sheet with rotating alignment, (FIG. 3B): A circular flat collector with 6 cm diameter was cut by Epilog Mini 24 laser cutter from an acrylic sheet ( 1/16″ thickness, part No. 8560K171, McMaster-Carr). The collector was taped onto a 10 mm steel bar, which sticks into the fiber stream without perturbing its air flow. The steel bar was inserted into the chuck of the spindle motor. The chuck was rotated every 5 minutes to obtain the scaffold with multiple angles. 16 mL polymer solution was collected at ˜8 cm distance.

Fiber tube with helical alignment, (FIGS. 3C-3E): A low contrast stiff Nylon MicroSwab (TX730, ITW Texwipe) with 0.9 mm diameter was used as the collector. The collector was rotated at 2,000 RPM with 450 helical angle. Spinning condition: 0.4 mL/min flow rate, ˜1.2 mL polymer solution, ˜20 cm collect distance.

Fibrous dual-ventricle model (FIGS. 5A-5D): Targets with the shape of left and right ventricle were designed in SolidWorks and were 3D printed. The multi-layered alignment was achieved through 4 steps. ˜2.5 ml polymer solution was deposited on each layer. Collection distance was ˜20 cm.

Single ventricle scaffold for cell seeding: Target of the left ventricle from the last experiment was used. ˜2 ml gelatin solution of 5 g/ml concentration was deposited on the target at ˜20 cm collection distance. The target was air dried for 1 hour to remove residual solvent. The gelatin scaffold was then immersed in an ethanol/water (95/5 vol %, 200 proof ethanol VWR and DI water) solution containing 0.016 M of 1-ethyl-3-(3-dimethylaminopropyl)carbodimide (EDC, Sigma-Aldrich), and 0.0064 M of N-hydroxysuccinimide (NHS, Sigma-Aldrich) for 24 h for cross-linking. Then the scaffold was washed by PBS three times to remove unreacted chemicals. The scaffold was then sterilized in 70% ethanol for 30 minutes and then placed under the UV Ozone for at least 7 minutes. Scaffolds were stored in de-ionized water for cell seeding.

### Scanning Electron Microscopy (SEM)

All samples were sputter coated with 10 nm of platinum/palladium (Pt/Pd) using a Quorum Sputter Coater (EMS 300T D, Quorum Technologies) to reduce charge accumulation. Then samples were imaged with a field emitting electron microscope (FESEM SUPRA 55, Zeiss) at a voltage of 5 kV.

To visualize the fiber orientation, a square area was cropped from the SEM image and Fast Fourier transform (FFT) was performed using ImageJ.

### X-Ray Micro Computed Tomography (CT)

X-ray micro computed tomography (μCT) imaging was completed on the Xradia 620 Versa 3D X-ray Microscope (Zeiss, Pleasanton, California, USA). The fiber sample was specially prepared on a low contrast stiff Nylon MicroSwab (TX730, ITW Texwipe) without any contrast agent staining. Micro-CT scanning was performed using a transmission tungsten X-ray source with tube voltage of 50 kV (photon energy levels ranging from 0 to 50 keV) and current of 90 uA. 4501 projection images were captured per sample on a 16-bit 2048×2048 20× objective detector with achievable voxel resolutions of 0.54 μm (sample type dependent). Total scanning time for each imaging is around 20 hours with 12 seconds for each exposure.

### Rat Heart Isolation and Cryosectioning

All animal experiments for the ventricular tissue immunostaining were approved by the Institutional Animal Care and Use Committee (IACUC) at Harvard University. Briefly, male Sprague Dawley rats (350-500 g) were anesthetized via inhalation of isoflurane (5% in O₂) and euthanized by cervical dislocation. The heart was removed and placed in cold (4° C.), oxygenated (95% 02:5% CO₂) Krebs buffer containing 10 mU/mL insulin and the following (mM): NaCl, 112.0; KCl, 4.7; KH₂PO₄, 1.2; MgSO₄, 1.2; CaCl₂), 2.5; NaHCO₃, 25.0; dextrose, 11.0 (pH 7.4). Prior to cryosectioning and immunostaining, the hearts were stored in PBS+30% sucrose solution overnight at 4° C., then transferred to 50% sucrose/50% optimal cutting temperature (OCT) watersoluble blend of glycols and resins for 24 hours at 4° C. They were then transferred to cryosectioning containers in 100% OCT and stored at 4° C. for 48 hours. Samples were frozen by partial immersion in 2-methylbutane which was, itself, partially immersed in liquid nitrogen. Frozen hearts were stored at −80° C. until cryosectioning by microtome (Leica). 10 μm-thick cross-sections were obtained that were transferred to glass microscope slides (Superfrost microscope slides, Sigma) and maintained at room temperature for 2 hours prior to storage at −80° C. until staining. For imaging, cross-sections of the heart at the equator, half-way from the base to apex were selected, where cross-sections included left and right ventricle walls but did not include heart valve features.

### NRVM Cell and Tissue Culture

To obtain cardiomyocytes, neonatal rat ventricular myocytes (NRVMs) were isolated from two-day-old neonatal s CRL: CD (Sprague-Dawley, SD) rat hearts.

NRVMs were seeded at a density of five million cells per ventricle. The NRVMs were cultured on the scaffolds with M199 culture media supplemented with 10% heat-inactivated fetal bovine serum (FBS), 10 mM HEPES, 0.1 mM MEM nonessential amino acids, 20 mM glucose, 2 mM L-glutamine, 1.5 M vitamin B12, and 50 U/mL penicillin. Samples were incubated at 37° C. and 5% CO₂. At 48 h post seeding the media was exchanged with M199 media containing 2% FBS and was exchanged again every 48 hours before testing.

The isolation of neonatal rat ventricular cardiomyocyte (NRVM) was performed based on the protocol used previously (Park S J, 2016; Feinberg A, 2007). The protocol was approved by Institutional Animal Care and Use Committee in Harvard University. Briefly, ventricles were surgically removed from 2-day old neonatal Sprague-Dawley rat pups (Charles River Laboratories, Wilmington, MA). The tissue was minced mechanically and rinsed in Hanks' balanced salt solution (HBSS; Thermo Fisher, Waltham, MA), followed by digestion in 1 mg/ml trypsin (MilliporeSigma, St. Louis, MO) in HBSS solution at 4° C. for 14 hours with gentle rocker agitation. The tissue was further homogenized in 1 mg/ml collagenase (MilliporeSigma, St. Louis, MO) in HBSS solution at 37° C. by four series of dissociation. Ventricular cardiomyocytes were collected from the solution by centrifugation and were filtered through a 40 μm cell strainer to remove undissociated clumps. By two series of 50-minute pre-plating, fibroblasts preferentially adhered to the substrate and cardiomyocytes were isolated and resuspended in M199 media (Life Technologies, Carlsbad, CA) supplemented with 10% (v/v) heat-inactivated fetal bovine serum (FBS; Life Technologies, Carlsbad, CA), 10 mM HEPES (Life Technologies, Carlsbad, CA), 1% (v/v) MEM non-essential amino acids (Life Technologies, Carlsbad, CA), 3.5 g/L glucose (MilliporeSigma, St. Louis, MO), 2 mM L-glutamine (Life Technologies, Carlsbad, CA), 2 mg/L vitamin B12 and 50 U/mL penicillin (Life Technologies, Carlsbad, CA). Cardiomyocytes were counted, adjusted to seeding density and ready to be seeded. After 24 hours incubation, the substrate was washed 3 times with PBS to remove non-adherent cells and provided with fresh media. After another 24 hours, we switched the media to M199 media supplemented as above but with 2% FBS to minimize growth of fibroblasts. Subsequently, the media was replaced every 48 hours until use, typically within 3 to 5 days, but no more than 6 days post seeding.

### Immunostaining

For immunostaining, 10 μm-thick cross-sections of heart or fiber scaffold with NRVM were permeabilized in 0.5% Triton-X100 for 20 min in PBS at 37° C., followed by 2 h incubation with 1:200 dilutions of mouse anti-sarcomeric a-actinin monoclonal primary antibody (Sigma-Aldrich, clone EA-53, catalogue number A7811-11UL). Samples were then washed and concurrently incubated with 1:200 dilutions of DAPI (Sigma-Aldrich), phalloidin conjugated to Alexa-Fluor 488 (Invitrogen) and goat anti-mouse secondary antibody conjugated to tetramethylrhodamine for 2 hours at room temperature. Imaging was performed using a Zeiss LSM 5 LIVE confocal microscope with a Plan-Neofluar 20×/1.3 oil objective.

### Optical Mapping Experiments

Calcium activities of the engineered 3D fibers structure were monitored with a calcium indicator, X-Rhod-1 (Invitrogen, Carlsbad, CA), using a modified tandem-lens macroscope that (Scimedia, Costa Mesa, CA) equipped with a high-speed camera (MiCAM Ultima, Scimedia, Costa Mesa, CA), a plan APO 0.63× objective, a collimator (Lumencor, Beaverton, OR), and a 200 mW Mercury lamp (X-Cite exacte, Lumen Dynamics, Canada), and a high-spatial resolution sCMOS camera (pco.edge, PCO AG) and 880 nm darkfield LED light were incorporated into the system. For Ca²⁺ imaging, an excitation filter with 580/14 nm, a dichroic mirror with 593 nm cut-off, and an emission filter with 641/75 nm (Semrock, Rochester, NY) were used. For dark field imaging, a dichroic mirror with 685 nm cut-off and long pass emission filter with 664 nm cut-off filter (Semrock, Rochester, NY) were added. Samples after 7 days culture were incubated with 2 μM X-Rhod-1 (Invitrogen) for 60 min at 37° C., rinsed, and incubated in dye-free media for an additional 15 min at 37° C. before recording. Prior to recording for the experiments, the culture media was replaced with Tyrode's solution (1.8 mM CaCl₂), 5 mM glucose, 5 mM Hepes, 1 mM MgCl₂, 5.4 mM KCl, 135 mM NaCl, and 0.33 mM NaH₂PO₄ in deionized water, pH 7.4, at 37° C.). The tissues were stimulated with a pulse generator. Point stimulation was applied using two platinum electrodes (Sigma-Aldrich) with 1 mm spacing with 12V amplitude and 10 ms duration. The platinum electrodes were located at 1.0 mm from the edge of “sample”. For each recording, Ca²⁺ and dark field images were acquired with 400 frames at a frame rate of over 10 s. Post-processing of data was conducted with custom software written in MATLAB (MathWorks) and MiCAM imaging software (MiCAM Ultima, Scimedia, Costa Mesa, CA). A spatial filter with 3×3 pixels was applied to improve the signal-noise ratio. Activation time of each pixel was calculated at the average maximum upstroke slope of multiple pulses of X-Rhod-1 signals over a 10 second recording window.

### Particle Imaging Velocimetry Measurements

Particle Imaging Velocimetry Measurements (PIV) measurements were conducted on a Zeiss Discovery.V12 stereomicroscope, with an HBO 100 fluorescent light source, using a Basler electric ACA2500-14UC USB 3.0 camera, recorded at a 10× magnification, at a frame rate of 30-50 frames per second, with resolution of 1920×1080 pixels. Prior to recording, samples were rinsed in fresh Phosphate Buffered Saline (PBS, 37° C.) and then placed in an imaging solution, comprised of Tyrode's Salt solution containing 10 μg/mL of suspended 1-5 μm green fluorescent beads (FMG—Green Fluorescent Microspheres, Cospheric, Santa Barbara, CA, USA). Samples were field stimulated with an IonOptix myopacer, using two parallel platinum electrodes positioned ˜30 cm apart, at a pacing frequency of 1-2 Hz, and 10 volts.

Velocity map reconstruction was done in python using custom software, based on the open source package OpenPIV. Fluorescent images were prepared for analysis using a rolling ball background subtraction (NIH ImageJ). Digital image cross-correlation was then performed on a frame-by-frame basis using OpenPIV, resulting in time evolved 2D velocity vector fields. Here outliers were filtered using spatial averaging. Any vector fields which exceeded 10× the standard deviation of the local vectors (3×3 grids), were replaced using the average value of the remaining vectors. This resulted in a time dependent velocity profile, which was phase averaged over the entire cycle to smooth out local fluctuations in performance. To phase average velocity fields, the sum of the longitudinal velocities was acquired, and fit using a least-squares regression to a sine wave. This provided the wavelength and phase-shift of each time series. Velocity vectors were then averaged over the full-cycle, providing a single averaged cycle. The resulting averaged velocity vectors were used to perform subsequent analysis.

### Mass Flux or Cardiac Output

As a simple metric pumping behavior, the instantaneous mass flux, N_{flux}, passing through the basal opening of the ventricle was determined based on the following equation:

N_{flux}=ρ_{f}∫_{∂S}u∂l

Where ρ_{f} is the fluid density (taken as ˜978 kg/m³ for Tyrode's solution), S is the region of analysis surrounding the opening, ∂S is the boundary of that region, ∂l is a line element of ∂S, and u is the fluid velocity. Cardiac output or 2D mass transfer, was then determined by integrating the mass flux over the systolic period, and is given in units of g/m. This returned a 2D metric, which by integrating over the whole z-depth of the ventricle opening, can be used to recover the total mass transferred, given in units of g.

### EQUIVALENTS

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step may be replaced with a plurality of elements or steps that serve the same purpose. Further, where parameters for various properties are specified herein for exemplary embodiments, those parameters may be adjusted up or down by 1/20th, 1/10th, ⅕th, ⅓rd, ½, etc., or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention. Further still, other aspects, functions and advantages are also within the scope of the invention.

The contents of all references, including patents and patent applications, cited throughout this application are hereby incorporated herein by reference in their entirety. The appropriate components and methods of those references may be selected for the invention and embodiments thereof. Still further, the components and methods identified in the Background section are integral to this disclosure and can be used in conjunction with or substituted for components and methods described elsewhere in the disclosure within the scope of the invention.

As may be recognized by those of ordinary skill in the pertinent art based on the teachings herein, numerous changes and modifications may be made to the above-described and other embodiments of the present disclosure without departing from the spirit of the invention as defined in the appended claims. Accordingly, this detailed description of embodiments is to be taken in an illustrative, as opposed to a limiting, sense.

Those skilled in the art will recognize, or be able to ascertain using no more than routine experimentation, many equivalents to the specific embodiments of the invention described herein. Such equivalents are intended to be encompassed by the following claims.

1. A method comprising:
providing or obtaining a three-dimensional tissue scaffold defining a lumen or a cavity, the tissue scaffold comprising one or more polymeric fibers each having a micron-scale or nanometer-scale diameter, at least some of the one or more polymeric fibers encircling the lumen or cavity at a helical angle with respect to a longitudinal axis of the lumen or cavity, at an azimuthal orientation with respect to the longitudinal axis of the lumen or cavity, or at both;
providing or obtaining cells growing on or in the three-dimensional tissue scaffold to form a three-dimensional tissue defining the lumen or the cavity;
affixing a first portion of the three-dimensional tissue to a support where the cells are grown on or in the three-dimensional tissue scaffold to form the three-dimensional tissue before the three-dimensional tissue is affixed to the support, or affixing a first end of the three-dimensional tissue scaffold to a support where the cells are grown on or in the three-dimensional tissue scaffold to form the three-dimensional tissue after the three-dimensional tissue scaffold is affixed to the support; and
measuring rotational displacement of at least a second portion of the three-dimensional tissue relative to the support over a period of time caused by functional activity of three-dimensional tissue or measuring strain of at least a second portion of the three-dimensional tissue relative to the support over a period of time caused by functional activity of the three-dimensional tissue.

2.-7. (canceled)

8. The method of claim 1, wherein measuring rotational displacement of at least the second portion of the three-dimensional tissue relative to the support over the period of time or measuring strain of at least the second portion of the three-dimensional tissue relative to the support over the period of time comprises obtaining images of at least the second portion of the three-dimensional tissue over the period of time.

9.-11. (canceled)

12. The method of claim 1, wherein the first portion of the three-dimensional tissue is disposed at a first end of the three-dimensional tissue and the at least the second portion of the three-dimensional tissue is disposed at a second end of the three-dimensional tissue opposite the first end of the three-dimensional tissue that is affixed to the support.

13. The method of claim 1, further comprising:
exposing the three-dimensional tissue scaffold to a solution containing fiducial markers such that the fiducial markers adhere to the three-dimensional tissue scaffold, or exposing the three-dimensional tissue to a solution containing fiducial markers that adhere to the three-dimensional tissue; and
wherein measuring rotational displacement of at least a second portion of the three-dimensional tissue relative to the support over a period of time or measuring strain of at least the second portion of the three-dimensional tissue relative to the support over the period of time comprises performing imaging of the fiducial markers in or on the at least a second portion of the three-dimensional tissue over the period of time.

14.-18. (canceled)

19. The method of claim 1, further comprising stimulating the three-dimensional tissue before, during, or before and during at least some of the measurement of the rotational displacement of at least the second portion of the three-dimensional tissue relative to the support over the period of time the measurement of the strain of at least the second portion of the three-dimensional tissue relative to the support over the period of time to cause a functional response in the three-dimensional tissue, to initiate the functional activity of the three-dimensional tissue, or to affect the functional activity of the three dimensional tissue.

20.-22. (canceled)

23. The method of claim 1, wherein the method is a method for identifying a compound that modulates tissue function; and
wherein the method further comprises:
contacting the three-dimensional tissue structure with a test compound, wherein measuring rotational displacement of at least a second portion of the three-dimensional tissue with respect to the support over a period of time or measuring strain of at least the second portion of the three-dimensional tissue relative to the support over the period of time includes:
measuring a first rotational displacement of the at least the second portion of the three-dimensional tissue relative to the support in the presence of the test compound over a first period of time or measuring a first strain of at least the second portion of the three-dimensional tissue relative to the support in the presence of the test compound over a first period of time; and
measuring a second rotational displacement of the at least the second portion of the three-dimensional tissue relative to the support in the absence of the test compound over a second period of time or measuring a second strain of at least the second portion of the three-dimensional tissue relative to the support in the absence of the test compound over a second period of time; and
comparing the first rotational displacement of at least the second portion of the three-dimensional tissue or the first strain of at least the second portion of the three-dimensional tissue with the second rotational displacement of the at least the second portion of the three-dimensional tissue or the second strain of at least the second portion of the three-dimensional tissue,
wherein a modulation of the first rotational displacement or the first strain in the presence of the test compound as compared to the second rotational displacement or the second strain in the absence of the test compound indicates that the test compound modulates tissue function, thereby identifying a compound that modulates tissue function.

24. The method of claim 1, wherein the method is a method for identifying a compound that is useful for treating or preventing a disease affecting tissue function, and
wherein the method further comprises:
contacting the three-dimensional tissue structure with a test compound, wherein measuring rotational displacement of the at least a second portion of the three-dimensional tissue with respect to the support over a period of time or measuring strain of at least the second portion of the three-dimensional tissue relative to the support over the period of time includes:
measuring a first rotational displacement of the at least the second portion of the three-dimensional tissue relative to the support in the presence of the test compound over a first period of time or measuring a first strain of at least the second portion of the three-dimensional tissue relative to the support in the presence of the test compound over a first period of time; and
measuring a second rotational displacement of the at least the second portion of the three-dimensional tissue relative to the support in the absence of the test compound over a second period time or measuring a second strain of at least the second portion of the three-dimensional tissue relative to the support in the absence of the test compound over a second period of time; and
comparing the first rotational displacement of at least the second portion of the three-dimensional tissue or the first strain of at least the second portion of the three-dimensional tissue with the second rotational displacement of the at least the second portion of the three-dimensional tissue or the second strain of at least the second portion of the three-dimensional tissue,
wherein a modulation of the first rotational displacement or the first strain in the presence of the test compound as compared to the second rotational displacement or the second strain in the absence of the test compound indicates that the test compound modulates tissue function, thereby identifying a compound useful for treating or preventing a disease affecting tissue function.

25. The method of claim 1, wherein the three-dimensional tissue is or includes a tissue engineered ventricle or a model of a ventricle, and wherein the method further comprises determining one or more ejection fractions for the three-dimensional tissue over time based on the measured rotational displacement of at least the second portion of the three-dimensional tissue relative to the support over the period of time, or based on the measured strain of at least the second portion of the three-dimensional tissue relative to the support over the period of time.

26.-33. (canceled)

34. A method comprising:
providing or obtaining a three-dimensional tissue scaffold defining a lumen or a cavity, the tissue scaffold comprising one or more polymeric fibers each having a micron-scale or nanometer-scale diameter, at least some of the one or more polymeric fibers encircling the lumen or cavity at a helical angle with respect to a longitudinal axis of the lumen or cavity, at an azimuthal orientation with respect to the longitudinal axis of the lumen or cavity, or both;
providing or obtaining cells growing on or in the three-dimensional tissue scaffold to form a three-dimensional tissue defining the lumen or the cavity;
exposing the three-dimensional scaffold to a medium including fiducial markers;
obtaining images of an area including or adjacent to an opening of the lumen or cavity over a period of time;
performing particle imaging velocimetry on fiducial markers in the images of the area including or adjacent to the opening of the lumen or cavity; and
determining a volume, mass flux, or velocity of fluid flow out of or into the opening due to or affected by a functional activity of the three-dimensional tissue based on the particle imaging velocimetry.

35. The method of claim 34, wherein the three-dimensional tissue is or includes a tissue engineered ventricle or a model of a ventricle, and wherein the method further comprises determining one or more ejection fractions for the three-dimensional tissue based, at least in part, on the determined volume, mass flux, or velocity of fluid flow out of or into the opening.

36.-39. (canceled)

40. The method of claim 34, wherein the fiducial markers are suspended in the medium.

41.-44. (canceled)

45. The method of claim 34, further comprising stimulating the three-dimensional tissue before, during, or before and during obtaining the images over the period of time, to initiate the functional activity of the three-dimensional tissue, or to affect the functional activity of the three-dimensional tissue.

46. The method of claim 34, wherein the method is a method of evaluating the functional activity of the three-dimensional tissue in response to the stimulation.

47. (canceled)

48. (canceled)

49. The method of claim 34, wherein the method is a method for identifying a compound that modulates tissue function;
wherein the method further comprises contacting the three-dimensional tissue structure with a test compound;
wherein obtaining images of the area including or adjacent to the opening of the lumen or cavity over a period of time includes:
obtaining first images of the area in the presence of the test compound over a first period of time; and
obtaining second images of the area in the absence of the test compound over a second period of time;
wherein determining a volume, mass flux, or velocity of fluid flow out of or into the opening due to or affected by a functional activity of the three-dimensional tissue based on the particle imaging velocimetry includes:
determining a first volume, mass flux, or velocity of fluid flow out of or into the opening during the first period of time; and
determining a second volume, mass flux, or velocity of fluid flow out of or into the opening during the second period of time; and
wherein the method further comprises comparing the first volume, mass flux, or velocity of fluid flow with the second volume, mass flux, or velocity of fluid flow, wherein a modulation of the first volume, mass flux, or velocity of fluid flow in the presence of the test compound as compared to the second volume, mass flux, or velocity of fluid flow in the absence of the test compound indicates that the test compound modulates the tissue function.

50. The method of claim 34, wherein the method is a method for identifying a compound that is useful for treating or preventing a disease affecting tissue function;
wherein the method further comprises contacting the three-dimensional tissue structure with a test compound;
wherein obtaining images of the area including or adjacent to the opening of the lumen or cavity over a period of time includes:
obtaining first images of the area in the presence of the test compound over a first period of time; and
obtaining second images of the area in the absence of the test compound over a second period of time;
wherein determining a volume, mass flux, or velocity of fluid flow out of or into the opening due to or affected by a functional activity of the three-dimensional tissue based on the particle imaging velocimetry comprises:
determining a first volume, mass flux, or velocity of fluid flow out of or into the opening during the first period of time; and
determining a second volume, mass flux, or velocity of fluid flow out of or into the opening during the second period of time; and
wherein the method further comprises comparing the first volume, mass flux, or velocity of fluid flow and the second volume, mass flux, or velocity of fluid flow, wherein a modulation of the first volume, mass flux, or velocity of fluid flow as compared to the second volume, mass flux, or velocity of fluid flow indicates that the test compound modulates tissue function, thereby identifying a compound useful for treating or preventing a disease affecting tissue function.

51.-60. (canceled)

61. The method of claim 1, wherein the three-dimensional tissue structure comprises one or more of a heart, an artery, a blood vessel, a lymph node, a lymphatic vessel, an intestine, and an inner layer of a tongue.

62. The method of claim 1, wherein the three-dimensional tissue scaffold includes:
a first layer in which a first portion of the one or more polymeric fibers encircle the longitudinal axis of the lumen at a first helical angle with respect the longitudinal axis of the lumen or cavity; and
a second layer in which a second portion of the one or more polymeric fibers encircle the longitudinal axis of the lumen or cavity a second helical angle with respect to the longitudinal axis of the lumen or cavity, wherein a difference between the first helical angle and the second helical angle falls is less than 90°.

63. (canceled)

64. (canceled)

65. The method of claim 1, wherein providing or obtaining the three-dimensional tissue scaffold comprises:
rotating a reservoir holding a material comprising a polymer about a rotation axis to eject at least one jet of material from at least one orifice defined by an outer sidewall of the reservoir;
directing at least one flow of gas through a portion of the reservoir radially inward of the outer sidewall, the at least one flow of gas directed from an upstream first end of the reservoir to a downstream second end of the reservoir during rotation of the reservoir and ejection of the at least one jet of the material to form at least one micron or nanometer dimension polymeric fiber, the at least one flow of gas entraining the at least one micron or nanometer dimension polymeric fiber and forming a focused fiber deposition stream of the at least one micron or nanometer dimension polymeric fiber in a first direction, the first direction having an orientation of within 45 degrees of the rotation axis of the reservoir; and
collecting the focused fiber deposition stream on a target surface that is being rotated about a second rotation axis, the target surface having a shape corresponding to at least a portion of the three-dimensional tissue scaffold.

66. (canceled)
